(19) United States
(12) Patent Application Publication
(10) Pub. No.: US 2009/0093964 A1
(43) Pub. Date: Apr. 9, 2009

(54) MULTI-DIMENSIONAL PARAMETER IDENTIFICATION METHOD AND DEVICE: APPLICATION TO THE LOCATION AND RECONSTRUCTION OF DEEP ELECTRICAL ACTIVITIES BY MEANS OF SURFACE OBSERVATIONS

(75) Inventors: Laurent Albera, Rennes (FR); Delphine Cosandier-Rimele, Rennes (FR); Isabelle Merlet, Liffre (FR); Fabrice Wendling, Thorigne-Fouillard (FR)

Correspondence Address: WESTMAN CHAMPLIN & KELLY, P.A., SUITE 1400, 900 SECOND AVENUE SOUTH, MINNEAPOLIS, MN, 55402, US

(73) Assignee: Universite de Rennes 1, Rennes Cedex (FR)

(21) Appl. No.: 12/094,078

(22) PCT Filed: Nov. 17, 2006
(86) PCT No.: PCT/EP2006/068636

§ 371 Date: Oct. 7, 2008

(30) Foreign Application Priority Data
Nov. 17, 2005 (FR) 0511668

(51) Int. Cl. G01V 3/38; A61B 5/0476

(52) U.S. Cl. 702/14; 702/19

(57) ABSTRACT

A method is provided for identifying multidimensional parameters of a plurality of P>1 sources of interest present in a predetermined multidimensional conductive environment by a plurality of observations (60) in a finite number of N≧1. The method includes using i) a factorisation of a problem matrix formulation, ii) the creation of a virtual network of the order 2q (q>1) sensors by using cumulants of order 2q from observations and iii) the concept of an extended deflation of order 2q taking into consideration the presence of potentially (but not entirely) correlated sources. The method and device can be used for electroencephalograpy, magnetoencephalography, geophysics and seismology.

### CROSS-REFERENCE TO RELATED APPLICATIONS

This Application is a Section 371 National Stage Application of International Application No. PCT/EP2006/068636, filed Nov. 17, 2006 and published as WO 2007/057453A1 on May 24, 2007, not in English.

### STATEMENT REGARDING FEDERALLY SPONSORED RESEARCH OR DEVELOPMENT

None.

### THE NAMES OF PARTIES TO A JOINT RESEARCH AGREEMENT

None.

### FIELD OF THE DISCLOSURE

The field of the disclosure is that of the acquisition and processing of representative signals of activities generated by a set of internal sources in a given multi-dimensional environment to be studied.

More specifically, the disclosure relates to a novel multi-dimensional parameter identification technique which makes it possible, among other things, to locate and reconstruct electrical activities, commonly referred to as sources, generated within a multi-dimensional environment, solely on the basis of observations acquired at certain points of said environment by means of a set of physical sensors.

Therefore, the disclosure is associated with a context and technical problem liable to be of interest to numerous disciplines for which said problem of identifying multi-dimensional parameters of sources of interest on the basis of observations is primordial and necessary for the improved understanding of internal phenomena in a studied environment.

These disciplines relate, as illustrative and non-limitative examples, equally well to the biomedical field, and more specifically human or animal electrophysiology, and the field of geophysics, for example to estimate the position of the epicenter and propagation of earthquakes.

### BACKGROUND OF THE DISCLOSURE

1. Specific Context of Electrophysiology

In the complementary fields of ElectroEncephaloGraphy (EEG) and MagnetoEncephaloGraphy (MEG), the post-synaptic activities of the cortical neurons may be recorded in a completely non-invasive manner, i.e. without needing to implant intracerebral electrodes.

EEG collects the surface electrical activity by means of sensors (recording electrodes) arranged in a standardized manner on the scalp and makes it possible to represent the progression over time of a difference in electric potential between each electrode and a common reference.

In parallel, MEG records the magnetic field (of very low amplitude) induced by cerebral electrical activity by means of ultrasensitive sensors arranged on a helmet covering the entire scalp.

In healthy subjects, these two techniques make it possible to record the specific spontaneous rhythms associated with normal physiological states (waking, sleeping, attentive states); they also make it possible to collect the responses induced by a given stimulation, which reflect the activation of the various cerebral structures involved during perceptual and/or cognitive processes.

Similarly, EEG and MEG recordings enable a clearer understanding of the mechanisms involved in the context of specific neurological conditions such as epilepsy, Alzheimer's disease or brain tumors.

In any case, the analysis of the neuronal bases of a normal complex cognitive system or a pathological dysfunction, such as an epileptic attack for example, requires the precise identification both of the participating cerebral regions and the temporal activation sequence between these regions.

Known functional imaging techniques (fMRI for functional magnetic resonance imaging, PET for Positron Emission Tomography, SPECT for Single Photon Emission Computed Tomography), which generally benefit from a high spatial resolution (of the order of one mm), have proven their effectiveness in the definition of the anatomical areas activated during cognitive operations. On the other hand, these methods remain severely limited in their ability to detect the temporal information on this activation as, at best, they provide a mean activation image over several hundred milliseconds.

Conversely, EEG and MEG, due to their excellent temporal resolution (<1 ms), enable a precise study of neuronal activation dynamics. However, the precise identification of the cerebral regions activated requires the use of mathematical tools that are both reliable and precise making it possible to solve the inverse problem, i.e. locating and reconstructing the sources of the cerebral activity in question solely on the basis of surface observations.

As illustrated in FIG. 1, the inverse problem in human electrophysiology is defined as the possibility, on the basis of simple surface recordings (electric potentials and/or magnetic fields) 11 of the cerebral activity from sensors 10 and using suitable head 12 and source models for the conduction medium in question, of identifying the cerebral regions 13 responsible for the EEG and/or MEG activities recorded. More specifically, the inverse problem in EEG and in MEG consists of estimating the parameters of the dipolar sources of the cerebral activity and reconstructing the associated time courses, solely on the basis of surface observations.

As a general rule, the resolution of the inverse problem in EEG and in MEG requires the definition of:
a source model, which should account for the spatial and temporal characteristics of the neuronal sources at the source of the cerebral electromagnetic activity; and
a conducting volume model, which should reproduce as well as possible the geometry and physical properties of all the constituents of the head;

and the resolution of the associated direct problem, which relates to characterizing the conduction of the activity of the sources within the conducting volume.

These different points are described in more detail below.

Note that the inverse problem is not specific to electrophysiology as it is also encountered, for example, in the field of seismology, when trying to estimate the epicenter and propagation of earthquakes, as described in the publication by A. TARANTOLA: “Inverse Problem Theory and Model Parameter Estimation”, republished by SIAM in 2004 and in the article by S. MIRON, N. LE BIHAN and J. I. MARS: “Vector-sensor MUSIC for polarized seismic sources localization” published in EURASIP Journal on Applied Signal Processing, vol. 10, pp. 74-84, October 2005. The above article describes in detail the specific context of seismic exploration, in particular direct problem formulation.
2. Source Model

In order to represent cerebral electromagnetic activity, the more commonly used source model is the dipolar model, which assimilates the activity of a small cortical zone to that of a current dipole, as represented in FIG. 2.

A distinction is made between two scenarios.

In the first scenario, the number of dipoles is assumed to be less than or equal to the number of surface observations (EEG or MEG activities). This is referred to as an overdetermined source mixture, or a “well-posed” problem. However, in practice, it is inaccurate to reduce the cerebral function, whether normal or pathological, to the activity of a restricted number of sources.

A second scenario is then taken into consideration, wherein the number of sources is assumed to be strictly greater than the number of surface observations. The source mixture is in this case said to be under-determined and the problem is “poorly posed”.

In the second scenario, it is important to make a distinction between the problem of locating the dipoles from that of reconstructing the deep electrical activities generated by same. In fact, while the second problem cannot theoretically be resolved in a single manner without adding and processing prospective information on the sources of interest, this is not the case for the first problem.

The inventors observed that this result is misunderstood by researchers in the biomedical field.
3. Conducting Volume Model

In addition to the specific source characteristics, the electric potential and the magnetic field recorded on a subject's scalp surface are also associated with the physical and geometric stress of the various tissues of the head. The head can be compared to a conducting volume which needs to account for differences in the various media (brain, cerebrospinal fluid, skull and skin). Therefore, the head is generally modeled using a set of three or four concentric layers, of different conductivities, representing the different tissues passed through when the signal from the sources reaches the skin surface.

The conductivities of each of these media may be considered to be isotropic, as described in the article by S. RUSH and D. A. DRISCOLL, “EEG electrode sensitivity—An application of reciprocity”, IEEE Transactions on Biomedical Engineering, vol. 38, pp. 15-22, January 1969, or anisotropic, as described in the article by J. C. De MUNCK and M. J. PETERS, “A fast method to compute the potential in the multisphere model”, IEEE Transactions on Biomedical Engineering, vol. 40, pp. 1166-1174, November 1993.

The simplest geometric model is the spherical model (FIG. 3.a) which compares the head to a set of concentric spheres, where each layer corresponds to a different tissue. The most commonly used model is a three-sphere model, representing a subject's brain 30, skull 31 and scalp 32, respectively.

However, the spherical model is only a rough approximation of the geometry of the head. Therefore, more realistic geometric models (FIG. 3.b) have been developed, and are constructed, for each subject, on the basis of anatomical MRI images. In fact, methods make it possible to extract, by means of MRI image segmentation, the contours of the three structures of interest which are the brain 33, the skull 34 and the scalp 35, and generate 3D meshings of these three surfaces.
4. Resolution of Direct Problem

Solving the direct problem with EEG and MEG consists of knowing how to calculate the electromagnetic field generated on the scalp surface by means of a configuration of known sources in the cerebral volume.

The application of the laws of physics such as Maxwell's equations, the load conservation law and the Biot-Savart law makes it possible to calculate the electric potential and the magnetic field created on the surface sensors, in view of the configuration of the intracerebral sources, and the geometry and conductivities of the various head tissues.

The choice of the electric potential and magnetic field calculation method will depend on the head model type. In the case of a spherical model (FIG. 3.a), it is possible to calculate analytically the electric potential and the magnetic field generated by a given dipole, as described in the articles by P. BERG and SHERG, “A fast method for forward computation of multiple-shell spherical head models”, Electroencephalography and Clinical Neurophysiology, vol. 90, no. 1, pp. 58-64, January 1994, and by J. SARVAS, “Basic mathematical and electromagnetic concepts of the biomagnetic inverse problems”, Physics in Medicine and Biology, vol. 32, pp. 11-22, 1987, respectively.

In the case of the abovementioned realistic models (FIG. 3.b), the calculation of the electric potential and the magnetic field is only feasible using known numerical methods from the prior art, i.e.:
the boundary element method (BEM), described clearly in the article by A. S. FERGUSON, X. ZHANG and G. STROINK, “A complete linear discretization for calculating the magnetic field using the boundary element method”, IEEE Transactions on Biomedical Engineering, vol. 41, pp. 455-459, 1994;
the finite element method (FEM), detailed in the article by Y. TAN, P. L. NUNEZ and R. T. HART, entitled “Finite-element model of the human head: scalp potentials due to dipole sources”, Med. Biol. Eng. Comput., vol. 29, no. 5, pp 475-481, 1991; and
the finite difference method (FDM), also described clearly in the article by L. LEMIEUX, A. McBRIDE and J. W. HAND, entitled “Calculation of electrical potentials on the surface of a realistic head model by finite differences” Phys. Med. Biol., vol. 41, no. 7, pp 1079-1091, 1996.

It should be added that J. C. MOSHER, R. M. LEAHY, and P. S. LEWIS, in their articles entitled “Matrix kernels for MEG and EEG source localization and imaging”, ICASSP 95, 1995 IEEE International Conference on Acoustics Speech and Signal Processing, vol. 5, Detroit, Mich., May 1995, pp. 2943-2946 and: “EEG and MEG: Forward solutions for inverse methods”, IEEE Transactions on Biomedical Engineering, vol. 46, no. 3, pp. 245-259, March 1999, proposed a common calculation mode based on i) a spatio-temporal matrix formulation of the direct problem, and ii) a factorization of said matrix formulation, thus separating the quasi-linear so-called nuisance parameters of interest, i.e. the orientation parameters of the sources, the non-linear parameters of interest, i.e. the location parameters of said sources and the linear parameters of interest, i.e. the time courses of said sources.
5. Known Solutions Resolving the Inverse Problem

Known Prior Art

Irrespective of the field of application, direct research on multi-dimensional parameters (for example dipole location and orientation) requires the resolution of a non-convex optimization problem. To this end, several approaches have emerged in the last thirty years.

The field of radiocommunications, for its part, has offered and continues to offer a panel of algorithms treating this problem. In telecommunications, the non-linear parameters are the angles of incidence of the sources on the receiving antenna and the nuisance parameters are polarizations of the sources which can only be estimated in the presence of a polarization diversity antenna.

We shall cite the best-known methods used to estimate the angles of incidence and polarization of the sources, and more generally the non-linear and quasi-linear parameters of the sources of interest.

One of the best-known methods is the MUSIC (MUltiple SIgnal Classification) method processing the second order statistics and the covariance matrix of the observations, described by R. O. SCHMIDT in the publications: “A signal subspace approach to multiple emitter location and spectral estimation”, a doctoral dissertation with Stanford University, November 1981 and “Multiple emitter location and signal parameter estimation”, IEEE Transactions On Antennas Propagation. vol. 34, no. 3, pp. 276-280, March 1986.

This method belongs to a large category of so-called subspace methods which also make use of the orthogonality between the vectorial space of the sources and that of the noise via second order statistics. This is made possible in the second order, during multi-sensor recordings, when the number of sources, referenced P, is strictly smaller than the number of observations, referenced N.

However, although Schmidt's MUSIC method makes it possible, in the presence of a polarization diversity antenna, to estimate the polarizations of the sources received, it does not make use of the possible factorization of the direct problem. In addition, although, in theory, the MUSIC method makes it possible to estimate both the non-linear parameters and the quasi-linear parameters, this is difficult to carry out in an operational context due to the calculation complexity involved.

It was necessary to wait for E. FERRARA et al. in “Direction finding with an array of antennas having diverse polarizations”, IEEE transactions on Antenna Propagation, vol. 31, pp. 231-236, March 1983, to offer MUSIC the possibility to carry it out in a practical context. In fact, E. FERRARA proposed a new version of MUSIC making use of factorization of the matrix formulation of the direct problem, and thus dissociated the estimation of the non-linear parameters (i.e. the current dipole positions in human electrophysiology) from that of the quasi-linear parameters (i.e. the orientations of said dipoles) of the sources, thus reducing the algorithm calculation cost considerably. In this case, E. FERRARA's algorithm renders the simultaneous estimation of non-linear and quasi-linear parameters feasible in a practical context.

Several years layer, sequential versions of MUSIC appeared, making use of the deflation concept in the second order. These versions include the RapMUSIC method, described in the article by J. C. MOSHER and R. M. LEAHY, “Source localization using Recursively Applied and Projected (RAP) MUSIC”, IEEE Transactions on Signal Processing, vol. 47, no. 2, pp. 332-340, February 1999. The latter particularly makes it possible to facilitate the location of several local optima, or in other words improve the estimation of the parameters of interest in the MUSIC metric, particularly when the size of the source space increases or when the non-linear parameters are very close between sources.

Unlike other sequential approaches, the RapMUSIC method is based on the work of E. FERRARA, thus offering the possibility of making use of a possible factorization of the matrix formulation of the direct problem in the presence of a polarization diversity antenna, for example, and more generally when the quasi-linear parameters are unknown and need to be estimated.

While the previous approaches only make use of second order statistics, and therefore the covariance matrix of the observations, B. PORAT and B. FRIEDLANDER decided to propose a version of MUSIC making use of fourth order statistics and therefore the quadricovariance matrix of the observations, as described in their article entitled “Direction finding algorithms based on high-order statistics”, IEEE Transactions on Signal Processing, vol. 39, no. 9, pp. 2016-2024, September 1991.

The method offers the benefit of enabling the resolution of a poorly posed inverse problem, and more precisely the processing of not more than P=N²−1 sources using only N observations.

Nevertheless, this method does not enable, on the other hand, the use of a possible factorization of the matrix formulation of the direct problem and is algorithmically different to the original version of MUSIC not only due to the use of high-order statistics, but also in that the algebraic structure of the quadricovariance matrix is different to that of the covariance matrix, and thus required the authors to modify the original version of MUSIC.

More recently, SATOSHI NIJIMA and SHOOGO UENO proposed a method, called UFO-MUSIC, making use of both fourth order statistics and a possible factorization of the matrix formulation of the direct problem, as described in the article “Universal Fourth Order Music Method: Incorporation of ICA into MEG Inverse Solution”, Third international conference on Independent Component Analysis and Blind Signal Separation, Dec. 9-12, 2001, San Diego, Calif., USA.

However, while the authors of this method claim to make use of fourth order statistics, they use them in a different form to that used by B. PORAT and B. FRIEDLANDER. In fact, they make use of one of more so-called contracted quadricovariance matrices wherein the algebraic structure is different to that of the quadricovariance matrix of B. PORAT and B. FRIEDLANDER. However, it is on the other hand equivalent to that of a covariance matrix, which enables them to us E. FERRARA's MUSIC algorithm or even the RapMUSIC method without modifying same when a single contracted quadricovariance matrix is used. When several matrices are used, the authors propose to estimate the signal space and the noise space by means of joint diagonalization of the various contracted quadricovariance matrices used. The MUSIC and RapMUSIC algorithms may then be used again without needing to modify them.

Moreover, it is important to mention the works by M. VIBERG and B. OTTERSTEN, particularly the WSF (Weighted Subspace Fitting) technique, described in their article entitled “Sensor array processing based on subspace fitting”, IEEE Transactions on Signal Processing, vol. 39, pp. 1110-1121, May 1991. It should be noted that this method, derived from a maximum probability type approach, gives performances, in terms of bias and variance, approximating in asymptotic terms those given by the Cramer-Rao bound.

The linear parameters, in other words the time courses of the sources, may be estimated, only in the overdetermined scenario, after estimating the non-linear, quasi-linear and nuisance parameters, particularly by reconstructing the transfer function linking the observations with the time courses of the sources. In fact, this makes it possible, among other things, to construct the ASF filter (Adapted Spatial Filter), described in P. CHEVALIER, “Optimal separation of independent narrow-band sources: Concept and Performances”, Signal Processing, Elsevier, vol. 73, pp. 27-47, 1999, which, applied to N observations, makes it possible to estimate and reconstruct the time courses of the P sources.

With respect exclusively to the location and reconstruction of intracerebral electrical activities, other algorithms have emerged. The reader may refer to the article by C. M. MICHEL, M. M. MURRAY, G. LANTZ, S. GONZALEZ, L. SPINELLI, and R. GRAVE DE PERALTA, “EEG source imaging”, Clinical Neurophysiology, vol. 115, no. 10, pp. 2195-2222, October 2004, for a review of the methods available.

In general, these algorithms try to explain, in the most optimal way possible, the scalp potentials via the intracerebral sources. Some of these methods are used to handle the under-determined scenario to estimate both the linear, quasi-linear and non-linear parameters, but require, however, the addition of prospective hypotheses on the sources of interest in order to obtain a single solution for the problem.
6. Drawbacks of the Known Solutions of the Prior Art

Most of the input direction estimation methods are based on second order statistics of the observations, in other words on the second order cumulants of the data acquired by means of the sensors used.

This involves, in an explicit or implicit manner, considering that the sources of interest are Gaussian. However, one drawback is that such a hypothesis is very strong, as in many applications the signals are generally non-Gaussian and also contain relevant statistical information, particularly in their cumulants of orders greater than 2.

Also, limiting oneself to the use of second order statistics may for this reason be limitative, as demonstrated by P. CHEVALIER, L. ALBERA, A. FERREOL and P. COMON in their article “On the virtual array concept for higher order array processing”, IEEE Transactions on Signal Processing, vol. 53, no. 4, pp. 1254-1271, April 2005, particularly in the presence of under-determined combinations of sources or Gaussian noise of unknown spatial consistency or when a specific level of resolution is required.

Therefore, second order methods have the major drawback of being limited in terms of performances and cannot be used, among other things, to handle under-determined source combinations.

In addition, with respect to the algorithm of B. PORAT and B. FRIEDLANDER, while it offers the possibility of handling up to P=N²−1 sources on the basis of only N observations, it does not make it possible, on the other hand, to reduce, by making use, for example, of a possible factorization of the matrix formulation of the direct problem, the calculation cost induced by multi-dimensional optimization. Consequently, an implementation of this algorithm is not feasible in an operational context when the quasi-linear parameters are unknown or need to be estimated. In addition, this method suffers from problems justifying the use of sequential approaches. Note that a progression of this method toward a sequential algorithm, which, as it makes use of a possible factorization of the direct problem, is in no way trivial because, as mentioned above, B. PORAT's quadricovariance matrix has a different algebraic structure to that of the covariance matrix. In addition, this progression has never been proposed or even suggested in the prior art. As regards the UFO-MUSIC method proposed by SATOSHI NIIJIMA and SHOOGO UENO, while the idea of jointly processing the information contained in the contracted quadricovariance matrices is interesting per se, the embodiment proposed by the authors is not justified. In fact, the joint diagonalization algorithm used requires a common orthonormed transition matrix for the various contracted quadricovariance matrices used, but the existence of such a solution is not demonstrated by the authors. For good reason, as, except in specific cases that cannot be used in an operational context, there is no guarantee that such a solution exists.

In addition, the approach proposed by SATOSHI NIIJIMA and SHOOGO UENO does not make it possible to resolve poorly posed inverse problems as encountered when the number of sources, P, is greater than the number of observations, N. This is essentially due to the fact that the authors prefer to make joint use of one or more linear combinations of matrix sections of the fourth order cumulant tensor rather than the tensor itself.

In addition, SATOSHI NIIJIMA and SHOOGO UENO assume in the implementation of their method that the sources are spatially statistically independent in the fourth order, which, in an operational context, is far from always being verified. For example, in epilepsy, two epileptic zones may have strongly correlated electrical activities, while having a distance of several centimeters from each other.

With respect to the WSF method, although offering very good performances, MUSIC type approaches offer a better compromise between the level of resolution of the estimation and the calculation cost induced.

As regards the methods arising from the biomedical field, and more specifically those making it possible to handle under-determined combinations of sources, they generally require the addition of hypotheses on the sources of interest to be studied. However, these hypotheses are sometimes purely mathematical and, if applicable, frequently disconnected from the physiology of the problem, which represents a major drawback in the processing and interpretation of the results obtained in this way.

Moreover, some of the methods in the biomedical field require reconstruction of the electrical activity at all points of the brain liable to be a solution of the inverse problem, which is very costly in terms of calculations, and therefore represents a major obstacle to the effective and relevant resolution thereof.

### SUMMARY

An aspect of the present disclosure relates to an identification method of multi-dimensional, linear, quasi-linear and non-linear type parameters, associated with a plurality of P≧1 sources of interest present in a predetermined multi-dimensional environment, by means of a plurality of observations in a finite number N≧1, obtained using physical sensors organized in the form of a network of sensors distributed at pre-defined points of said environment.

According to an embodiment of the invention, the physical sensors being organized in the form of a network of sensors distributed at predefined points of said environment, the identification method advantageously comprises at least the following steps:
recording of physical measurements making it possible to produce at least one vector of N observations generated by a mixture of linear parameters, representative of P sources of interest, and an additional noise;
construction, on the basis of said at least one observation vector, of a 2q (q≧2) order statistical matrix of the observations, said matrix being of the size (N^{q}×N^{q}), and for q≧2, of a different algebraic structure to that of the covariance matrix;
estimation of at least one first multi-dimensional parameter of said P sources of interest by means of the estimate of at least one second multi-dimensional parameter, so as to analyze and process a greater number of internal sources of interest in said environment under study, on the basis of observations acquired at certain points of said environment by means of a more limited number of physical sensors.

Therefore, an embodiment of the invention is based on a novel and inventive analysis and processing approach of representative data of internal sources of interest in a previously defined multi-dimensional environment. It represents a powerful data processing tool, offering a user the opportunity to increase the opening and increase the resolution of the sensor network using so-called virtual sensors obtained by using 2q (q≧2) order cumulants. This makes it possible to i) acquire a markedly improved estimation of the parameters of interest particularly in the presence of partially correlated sources, ii) estimate the non-linear and nuisance parameters in an under-determined context, and iii) be insensitive to the presence of a Gaussian noise of unknown spatial consistency.

Preferentially, the method according to an embodiment of the invention implements a location and reconstruction step of the electrical activity generated by the plurality of P≧1 sources of interest, representative of electric current sources, modeled in the form of electric current dipoles, referred to as dipolar sources, when said predetermined multi-dimensional environment is a conducting environment, the location and reconstruction steps accounting for the plurality of the finite number of N observations.

Advantageously, the linear, quasi-linear and non-linear parameters are respectively representative of the time courses or dipolar moments, the orientation and position parameters of each of the electric current sources.

Advantageously, for any time k, the observation vector of the length N is expressed in the following form: x(k)=A(Θ)s(k)+v(k) where:
s(k) is a vector, of the size (P×1), representative of the linear parameters corresponding to the time courses of said P sources of interest, which are non-Gaussian and potentially (but not completely) correlated according to said at least one first multi-dimensional parameter;
A(Θ) is an instantaneous mixture matrix, of the size (N×P), where Θ={Θ₁ . . . , Θ_{P}} is the set of the P vectors of quasi-linear and non-linear parameters of the sources of interest and where each of the P column vectors of A(Θ) is broken down in the form: α(Θ_{P})=G(ρ_{P})Φ_{P} where ρ_{P} and Φ_{P} represent respectively the non-linear parameters, on one hand, and quasi-linear parameters, on the other, associated with the p-th source of interest, the mixture matrix defining a transfer function between the P sources of interest and the N observations, and:
v(k) is the vector, of the size (N×1), of the additional noise, independent from the sources of interest.

Preferentially, the method according to an embodiment of the invention also comprises at least one step i) consisting of estimating the 2q order cumulants Cᵢ_{. . . i}_{_{q}}_{,x}ⁱ^{_{q+1}}^{. . . i}^{_{2q}} on the basis of the K samples x(k), ii) consisting of selecting the suitable matrix arrangement for which the estimated 2q order statistical matrix, of the size (N^{q}×N^{q}), will be referenced Ĉ_{2q,x}^{l}^{ₒₚₜ};

Advantageously, the method according to an embodiment of the invention also comprises at least one estimation step of the rank of said matrix Ĉ_{2q,x}^{l}^{ₒₚₜ}, and the number P of sources involved.

Also advantageously, the method according to an embodiment of the invention comprises at least one decomposition step into eigenvalues of the matrix Ĉ_{2q,x}^{l}^{ₒₚₜ} and a construction step of a cost function, referred to as a 2q order pseudo-spectrum or pseudo-polyspectrum, along with a minimization step of said cost function to estimate each of said \hat{P} vectors of quasi-linear and non-linear parameters associated with each of said \hat{P} sources of interest, where \hat{P} is the estimate of P.

Preferentially, the cost function is expressed in the form

$${\hat{J}}_{4} ( ρ ) = \frac{det \{ {{G}_{q}^{l} ( ρ )}^{H} {\hat{Π}}_{q , v}^{l} {G}_{q}^{l} ( ρ ) \}}{det \{ {{G}_{q}^{l} ( ρ )}^{H} {G}_{q}^{l} ( ρ ) \}} ,$$

where:
\hat{π}_{q,v}^{l}^{ₒₚₜ}=(Ê_{2q,v}^{l}^{ₒₚₜ})^{H} Ê_{2q,x}^{l}^{ₒₚₜ}| is a matrix operator, referred to as the 2q order noise projector, where ê_{2q,v}^{l}^{ₒₚₜ} is the matrix of the orthonormed eigenvectors associated with the null eigenvalues of said matrix Ĉ_{2q,x}^{l}^{ₒₚₜ};

$${G}_{q}^{l} ( ρ ) {=}^{def} {G ( ρ )}^{⊗ q - l} ⊗ {G ( ρ )}^{* ⊗ l}$$

where G(ρ) is the function gain matrix of the non-linear parameter vector ρ of the size (N×L), where L is the length of the quasi-linear parameter vector Φ.

Advantageously, the method according to an embodiment of the invention also implements a cost function minimization step, performed on the basis of a 2q (q>1) order deflation method representative of recursive estimation of each of the \hat{P} vectors of quasi-linear and non-linear parameters associated with each of the \hat{P} sources of interest.

Preferentially, the p-th step (1≦p≦P) of the recursive procedure comprises at least one of the following steps:
determination of the global minimum cost function, where the estimate is referenced \hat{ρ}_{ξ(P)};
calculation of a vector \hat{φ}_{q}^{l}^{ₒₚₜ} (\hat{ρ}_{ξ(P)}) taking the eigenvector corresponding to the minimum eigenvalue of the matrix ┌G_{q}^{l}^{ₒₚₜ}(\hat{ρ}_{ξ(P)})^{H}G_{q}^{l}^{ₒₚₜ}(\hat{ρ}_{ξ(P)})┐⁻¹ G_{q}^{l}^{ₒₚₜ}(\hat{ρ}_{ξ(P)})^{H} π_{q,v}^{l}^{ₒₚₜ}G_{q}^{l}^{ₒₚₜ}(\hat{ρ}_{ξ(P)});
extraction of a vector \hat{φ}_{ξ(P)} representing an estimate of the nuisance quasi-linear parameter vector Φ_{ξ(1)}, on the basis of the vector \hat{φ}_{q}^{l}^{ₒₚₜ} (\hat{ρ}_{ξ(P)});
in the event at least one source remaining wherein the quasi-linear and non-linear parameters have not yet been identified, i) construction of a vector

$$a ( {\hat{θ}}_{ξ ( p )} ) {=}^{def} G ( {\hat{ρ}}_{ξ ( p )} ) {\hat{φ}}_{ξ ( p )} ,$$

and ii) calculation of a matrix \hat{Σ}_{ξ(P)}^{q,l}^{ₒₚₜ} of the size (N^{q}×N^{q}) accounting for a replacement of said vector α(θ_{ξ(P)}) by said vector α(\hat{θ}_{ξ(P)}) and iii) reallocation of the variables according to the following functions:

$$\hat{P} := \hat{P} - 1 ;$$

$${\hat{G}}_{q}^{{l}_{opt}} ( ρ ) := {\hat{Σ}}_{ξ ( 1 )}^{q , {l}_{opt}} {\hat{G}}_{q}^{{l}_{opt}} ( ρ ) ;$$

$${\hat{C}}_{2 q . x}^{{l}_{opt}} := {\hat{Σ}}_{ξ ( 1 )}^{q , {l}_{opt}} {{\hat{C}}_{2 q / x}^{{l}_{opt}} ( {\hat{Σ}}_{ξ ( 1 )}^{q , {l}_{opt}} )}^{H} ;$$

Preferentially, the extraction step of a vector \hat{φ}_{ξ(P)} comprises the following sub-steps:
extraction of M=N^{q-2} vectors \hat{b}_{ξ(P)}^{q,l}^{ₒₚₜ} (m) of the size (N²×1);
conversion of the vectors into M matrices \hat{B}_{ξ(P)}^{q,l}^{ₒₚₜ} (m) of the size (N×N);
calculation of a common eigenvector for the M matrices of the set \hat{Δ}_{ξ(P)}^{q,l}^{ₒₚₜ} associated with the largest eigenvalue.

Also preferentially, if the number of sources of interest is less than the number of physical sensors, said method implements an ASF (“Alternating Sequential Filtering”) filter construction step defined by the formula Ŵ=[Ĉ_{2.x}⁰]⁻¹ A(\hat{Θ}), where A (\hat{Θ}) is the mixture matrix reconstructed from the estimation \hat{Θ} of said quasi-linear and non-linear parameters of the sources of interest, in order to estimate the linear parameters thereof.

An embodiment of the invention also relates to an identification device of multi-dimensional parameters of sources of interest characterized in that it comprises a processor suitable for implementing steps of the so-called linear, quasi-linear and non-linear multi-dimensional parameters associated with a plurality of P≧1 sources present in a predetermined multi-dimensional environment by means of a plurality of observations in a finite number N≧1, as described above.

Such a device may be integrated, as an illustrative and non-limitative example, in any type of acquisition device of representative data of deep sources of interest, on the basis of surface information captured by a set of sensors, i.e. seismograph type devices in the field of seismology or electroencephalograph and/or magnetoencephalograph type devices in the neurological field.

An embodiment of the invention also relates to a computer program product downloadable from a communication network and/or stored on a computer-readable medium and/or executable by a microprocessor, such as a program comprising program code instructions for the implementation of the steps of the identification method of multi-dimensional parameters, of the linear, quasi-linear and non-linear type, associated with a plurality of P≧1 sources of interest present in a predetermined multi-dimensional environment, by means of a plurality of observations in a finite number N≧1, as described above.

Finally, an embodiment of the invention also relates to the application of the abovementioned identification method of multi-dimensional parameters, of the linear, quasi-linear and non-linear type, associated with a plurality of P≧1 sources of interest present in a predetermined multi-dimensional environment, by means of a plurality of observations in a finite number N≧1, in the fields belonging the group comprising:
electroencephalography;
magnetoencephalography;
geophysics;
seismology.

It is understood that an embodiment of the invention may be applied to any other field requiring the identification of specific multi-dimensional parameters for sources of interest in an environment under study, solely on the basis of information acquired at certain points of said environment, once the observation model (or direct problem) allows a matrix expression dissociating the non-linear parameters from the quasi-linear parameters of said sources of interest.

### BRIEF DESCRIPTION OF THE DRAWINGS

Other characteristics and advantages will emerge more clearly on reading the following description of a preferential embodiment of the invention, given as an illustrative and non-limitative example, with reference to the appended figures, wherein:

FIG. 1, already discussed with respect to the state of the art illustrates the inverse problem resolution principle in electrophysiology;

FIG. 2 illustrates the source model used to represent neuronal activity;

FIG. 3 gives two examples of conducting volume models that can be used in EEG and in MEG within the scope of an embodiment of the invention;

FIG. 4 shows the Berg and Scherg approximation principle to calculate the surface electric potential within the scope of a spherical head model according to FIG. 3;

FIGS. 5 and 6 are organization charts showing the main steps of the method according to an embodiment of the invention respectively.

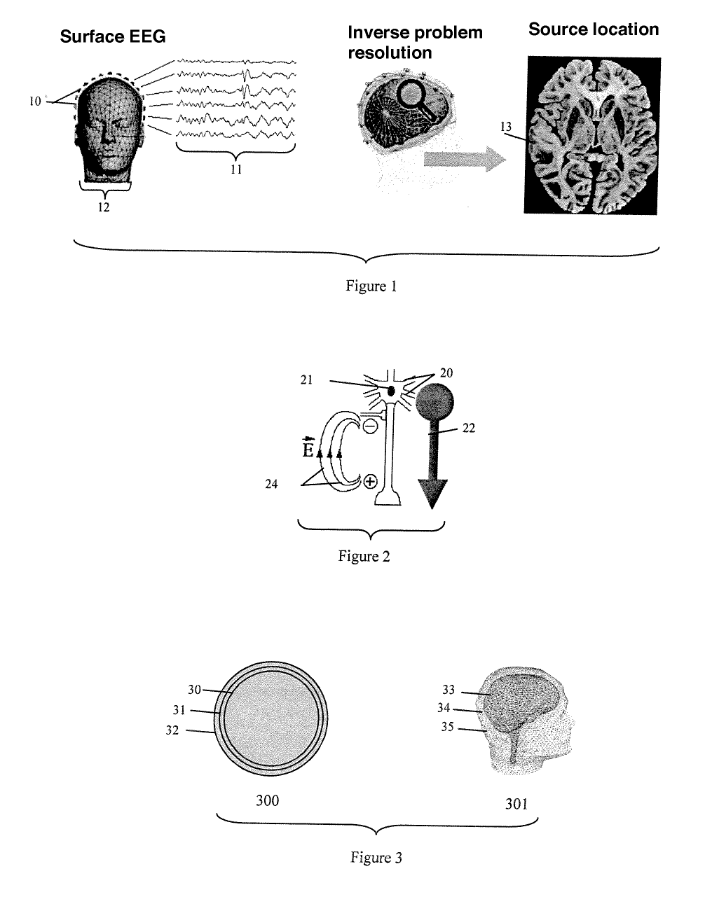

### DETAILED DESCRIPTION OF ILLUSTRATIVE EMBODIMENTS

The various figures are discussed below through the detailed description of an embodiment of the invention.

As of now, in relation to FIG. 2, if an excitatory synapse on the dendrites 20 of a cortical pyramidal neuron 21 is taken into consideration, the synaptic activation induces on the post-synaptic membrane a depolarization 22 comparable to a current input. This massive input of ions is counterbalanced by current outputs 24 downstream from this point, along the membrane. An activated neuron may, as a result, be compared to a group of positive charges separated by a small distance, i.e. at a current dipole. The extracellular currents 24 and, as a result, the potential fields established between positive and negative regions are the source of the EEG activities collected on the surface.

In addition, FIG. 3 illustrates in the left part thereof a spherical head model (300) consisting of three concentric layers, representing the brain 30, skull bone 31 and skin of the scalp 32. The same figure, in the right part thereof, taken from the doctoral dissertation by S. BAILLET (1998), illustrates a realistic geometric head model (301) consisting of 3 media (brain 33, skull 34 and scalp 35) and based, for each patient, on anatomical MRI image segmentation.
1. Description of a Preferred Embodiment of the Invention

The description of the embodiment of the invention is given here in the field of EEG, and more specifically the location/reconstruction of intracerebral electrical activities on the basis of data measured on the scalp surface. This description serves merely as an illustrative and non-limitative example of the invention. It is naturally possible to apply any disclosure from this embodiment described to any other field of application (for example to MEG or to geophysics) requiring at least calculation cost the estimation of specific linear, quasi-linear and non-linear parameters for internal sources of interest in a multi-dimensional environment, on the basis of mere observation data acquired at certain points of said environment, including in contexts where the number of sources of interest is potentially greater than the number of data measured, and the sources are potentially (but not completely) consistent. To be able to apply this embodiment to MEG or to seismic exploration, it is necessary to acknowledge the specific direct problem formulation for these two fields of application, Such information is accessible in the publication by BIN HE entitled “Modeling and Imaging of Bioelectrical Activity. Principles and Applications” published in 2004 by KLUMER ACADEMIC/PLENUM PUBLISHERS, NEW YORK with respect to MEG, and in the article by S. MIRON, N. LE BIHAN and J. L. MARS: “Vector-sensor MUSIC for polarized seismic sources localization” published in EURASIP Journal on Applied Signal Processing, vol. 10, pp. 74-84, October 2005 with respect to seismic exploration.
2. Hypotheses and Signal Modeling

A set of K N-dimensional vectors x(k) is observed. Each column vector x(k) contains the N electric potential differences obtained at the time k using N+1 surface sensors arranged on the patient's scalp. It is then assumed that, for any time k, the vector x(k) observes the following mathematical model:

x(k)=A(Θ)s(k)+v(k) (equation 1)

where s(k) is a vector of the time courses of the P sources, which are non-Gaussian and potentially (but not completely) correlated, A(Θ) is the instantaneous mixture matrix, of the size (N×P), where Θ={Θ₁ . . . , Θ_{P}} refers to the set of the quasi-linear and non-linear parameters associated with the P sources, i.e. for the application described, the set of parameters associated with source position and orientation. For its part, v(k) is the noise vector, assumed to be Gaussian and wherein the components are statistically independent from the time courses of the sources.

As mentioned above, the P sources may be correlated. It is assumed therefore that they may be divided into J groups, such that the sources from the same group are correlated, while the sources from different groups are statistically independent.

The term Pⱼ is used to reference the number of sources of the j-th group, and

$$P = {\sum }_{j = 1}^{J} {P}_{J} .$$

Note that the scenario where J=P corresponds to that where the P sources are statistically independent whereas the scenario where J=1 corresponds to that where they are all correlated.

The source vector s(k)^{T} is thus written as the concatenation of J vectors sⱼ(k)^{T}. It can therefore be stated that two components of s(k) are independent if and only if they are associated with two vectors sⱼ(k)^{T} and s_{j′}(k)^{T}, such that 1≦j≠j′≦J.

The combination matrix A(Θ) is also expressed as the concatenation of J matrices Aⱼ(Θ)ⱼ, of the size (N×Pⱼ), each corresponding to a group of dependent sources. Hereinafter, for simplification purposes, the notations Θ and Θⱼ will be omitted.

Within the scope of the location of sources with EEG, each column vector α(θ_{P}) of the matrix A(Θ) may be expressed as the product of a gain matrix G(θ_{P}), of the size (N×3), and a quasi-linear (or nuisance) parameter vector Φ_{P}, associated with the orientation of the p-th source:

∀p, 1≦p≦P, α(θ_{P})=G(θ_{P})Φ_{P} (equation 2)

The term θ_{P}=[ρ_{P}^{T} Φ_{P}^{T}]^{T} is used to reference the vector of the non-linear and quasi-linear parameters of the p-th source, where ρ_{P} is the vector of the non-linear position parameters and Φ_{P} the vector of the quasi-linear orientation parameters.

It is important to note at this stage that, once an application can be based on the same model as that defined by the equations (1) and (2), the methods and devices according to the invention are applicable.

The three-layer spherical head model will now be described, according to FIG. 3.a, in order to enable the implementation thereof. Each layer 30, 31, 32 represents a different tissue, having conductivity σⱼ (1≦j≦3) assumed to be homogenous and isotropic. All the current sources are confined in the sphere 30 representing the brain. They are represented by P current dipoles according to FIG. 2.

The p-th dipole is characterized by its position ρ_{P}, its orientation Φ_{P}, and its time course sₚ(k) at the time k.

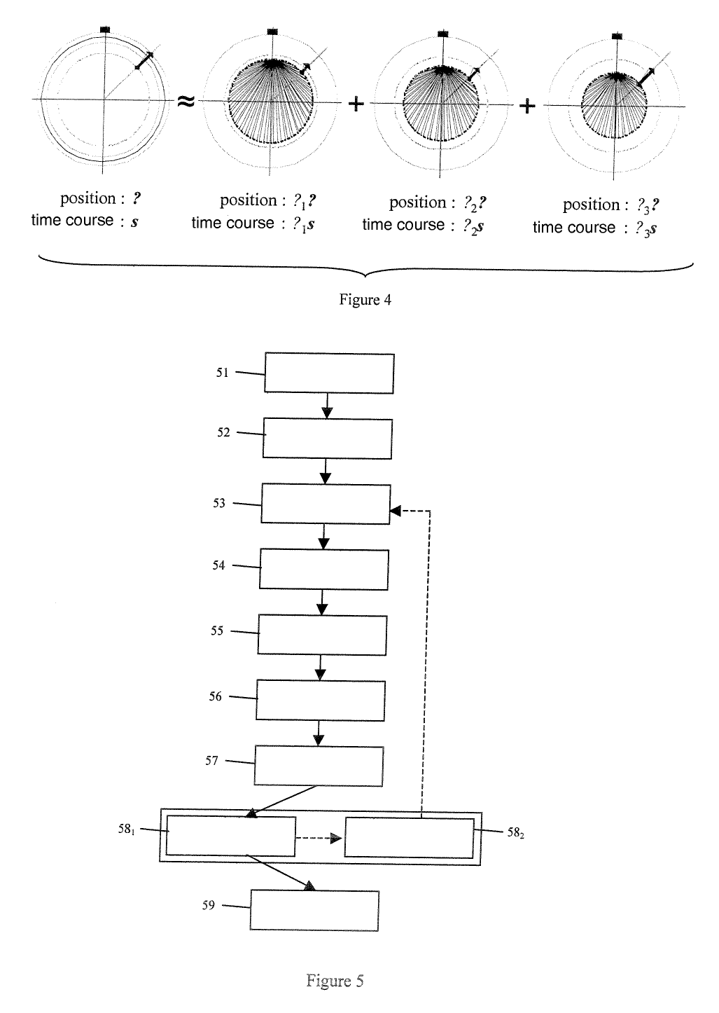

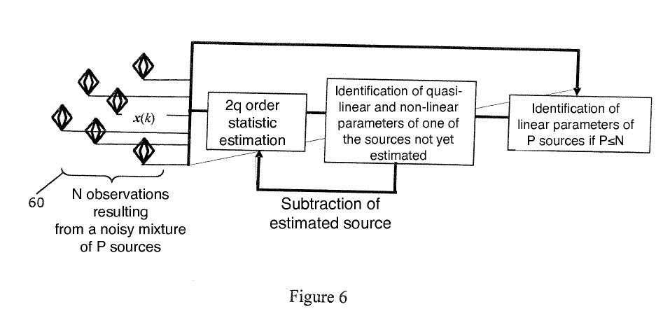

The contribution of the p-th dipole to the N surface electric potential differences via the gain matrix G(ρ_{P}), which in the case of a spherical head model, may be defined as follows, using the approximation of P. BERG and M. SHERG, presented in their article “A fast method for forward computation of multiple-shell spherical head models” Electroencephalography and Clinical Neurophysiology, vol. 90, no. 1, pp. 58-64, January 1994, and illustrated in FIG. 4:

$$G ( ρ p ) = {V [ \begin{matrix}{\sum }_{j = 1}^{3} {λ}_{j} h ( {r}_{1} , {μ}_{j} {ρ}_{p} ) , … , \\ {\sum }_{j = 1}^{3} {λ}_{j} h ( {r}_{N + 1} , {μ}_{j} {ρ}_{p} )\end{matrix} ]}^{T} \qquad ( equation 3 )$$

where V is a so-called switch matrix (as per J. C. MOSHER, R. M. LEAHY, and P. S. LEWIS, “Matrix kernels for MEG and EEG source localization and imaging”, ICASSP 95, 1995 IEEE International Conference on Acoustics Speech and Signal Processing, vol. 5, Detroit, Mich., May 8-12 1995, pp. 2943-2946) making it possible, when the potentials are recorded by N+1 sensors, to subtract the value collected by one of the sensors serving as a reference, from the set of values from the N other sensors, and thus obtain N potential differences.

In fact, the potential created by a dipole in a homogenous and isotropic three-sphere medium may be approximated by the sum of the potentials created by three dipoles, each dipole being positioned in a single homogenous and isotropic sphere (see FIG. 4). The three “unique” spheres are identical to the outer sphere (scalp) of the three-layer model (same radius, same conductivity). The three dipoles are oriented in the same way as the “original” dipole, their positions and their time courses are proportional to those of the original dipole (proportionality coefficients λᵢ for the time courses, μᵢ for the positions).

The vector h(r,ρ), of the size (N×3) is given by the following expression:

h(r,ρ)=[(c₁−c₂(r^{H}ρ))ρ+c2∥ρ∥²r] (equation 4)

where the parameters c₁and c₂ are defined by:

$${c}_{1} = \frac{1}{4 π σ {‖ ρ ‖}^{2}} ( 2 \frac{{( r - ρ )}^{H} ρ}{{‖ r - ρ ‖}^{3}} + \frac{1}{‖ r - ρ ‖} - \frac{1}{‖ r ‖} ) \\ {c}_{2} = \frac{1}{4 π σ {‖ ρ ‖}^{2}} ( \frac{2}{{‖ r - ρ ‖}^{3}} + \frac{‖ r - ρ ‖ + ‖ r ‖}{‖ r ‖ F ( r , ρ )} ) \qquad ( equation 5 )$$

where σ is the conductivity of the outer sphere (scalp) and:

F(r,ρ)=∥r−ρ∥(∥r∥ ∥r−ρ∥+∥r∥²−ρ^{H}r)| (equation 6)

Note that {λ₁,λ₂,λ₃,μ₁,μ₂,μ₃} forms the set of “Berg” parameters defined by P. BERG and M. SHERG, in their article entitled “A fast method for forward computation of multiple-shell spherical head models” Electroencephalography and Clinical Neurophysiology, vol. 90, no. 1, pp. 58-64, January 1994, and may be obtained, as per Z. ZHANG, as described in the article “A fast method to compute surface potentials generated by dipoles within multilayer anisotropic spheres”, Physics in Medicine and Biology, vol. 40, no. 3, pp. 335-349, March 1995, by minimizing the following quantity:

$$Δ = {\sum }_{n = 2}^{{N}_{max}} {( \frac{{R}_{1}}{{R}_{3}} )}^{2 n - 2} {( \begin{matrix}{f}_{n} - {f}_{1} {μ}_{1}^{n - 1} - \\ {\sum }_{j = 2}^{3} {λ}_{j} ( {μ}_{j}^{n - 1} - {μ}_{1}^{n - 1} )\end{matrix} )}^{2} \qquad ( equation 7 )$$

where the number of terms Nₘₐₓ should be sufficiently large to ensure satisfactory calculation precision (for example Nₘₐₓ=200), σⱼ and Rⱼ (1≦j≦3) are the conductivities and radii of the three spheres, respectively, and:

$$∀ n , {f}_{n} = \frac{n}{n {m}_{22} + ( 1 + n ) {m}_{21}} \qquad ( equation 8 )$$

The coefficients m₂₁, and m₂₂ are thus obtained as follows:

$$[ \begin{matrix}{m}_{11} & {m}_{12} \\ {m}_{21} & {m}_{22}\end{matrix} ] = \frac{1}{{( 2 n + 1 )}^{2}} {\prod }_{k = 1}^{2} [ \begin{matrix}n + ( n + 1 ) \frac{{σ}_{k}}{{σ}_{k + 1}} & ( n + 1 ) ( \frac{{σ}_{k}}{{σ}_{k + 1} - 1} ) {( \frac{{R}_{3}}{{R}_{4}} )}^{2 n + 1} \\ n ( \frac{{σ}_{k}}{{σ}_{k + 1} - 1} ) {( {R}_{k} / {R}_{3} )}^{2 n + 1} & ( n + 1 ) + n ( \frac{{σ}_{k}}{{σ}_{k + 1}} )\end{matrix} ] \vert$$

where the product matrices are not commutative.

Note that Mosher et al. also gave in: “EEG and MEG: Forward solutions for inverse methods”, IEEE Transactions on Biomedical Engineering, vol. 46, no. 3, pp. 245-259, March 1999, the expression of the gain matrix G(ρ_{P}) associated with the calculation of the magnetic fields in a spherical head model, along with a similar matrix formulation of the direct problem in EEG and MEG in the case of realistic head models (more precisely for the boundary element method (BEM)), as represented in FIG. 3.b.
3. 2q Order Statistics

The 2q order statistics of the observations are taken into consideration. For any k, the 2q order cumulant of a vector x(k) is defined as follows:

Cⁱ^{^{Q+1}}^{. . . i}^{^{2q}}ᵢ_{_{1 . . .}} ᵢ_{_{q}}_{,x}(k)=Cum{xᵢ_{₁}(k), . . . , xᵢ_{_{q}}(k),, xᵢ_{_{q+1}}(k)*, . . . , xᵢ_{_{2q}} (equation 10)

This statistical value is calculated on the basis of 2q components of the vector x(k), taking into consideration q conjugated terms xᵢ(k) and q non-conjugated terms.

It should be noted that if the sources of interest and the noise are stationary, the 2q order statistics are not dependent on the time index k, and may be referenced Cᵢ_{₁} _{. . . i}_{_{q}}_{,x}ⁱ^{_{q+1}} ^{. . . i}^{_{2q}}.

On the other hand, if the sources are cyclostationary, cycloergodic and potentially non-centered, other quantities must be used such as those defined in the article by L. ALBERA et al. entitled “Blind Identification of Overcomplete Mixtures of sources (BIOME)”, Linear Algebra Applications, Special Issue on Linear Algebra in Signal and Image Processing, vol. 391C, pp. 3-30, November 2004.

Hereinafter, for simplification purposes, the study will be limited to the stationary case. The non-stationary case may naturally be envisaged using an embodiment of the invention.

It is possible, by means of the method according to an embodiment of the invention, to arrange the set of 2q order statistics of the vector x(k) in a Hermitian matrix C_{2q,x}, of the size (N^{q}×N^{q}), referred to as the 2q order statistical matrix.

There are several methods for arranging these 2q order statistics in the matrix C_{2q,x}. However, only a finite number, referenced q₀, of arrangements makes it possible to obtain different results in terms of resolution and the number of sources handled. This number q₀ is generally a function of the number q mentioned above. However, note that, for observations at real values, such as for EEG, q₀=1.

These q₀ arrangements are indexed according to the integer l (0≦l≦q₀−1) and correspond respectively to q₀ 2q order statistical matrices, referenced C_{2q.x}^{l}. The (I₁^{l},I₂^{l})-th element (1≦I₁^{l},I₂^{l}≦N^{q}) of the matrix C_{2q,x}^{l} is in this case given by the following expression:

C_{2q,x}^{l}(I₁^{l},I₂^{l})=Cᵢ_{₁} _{. . . i}_{_{q}},xⁱ^{_{q+1}} ^{. . . i}^{_{2q}} (equation 11)

where for each number l such that 0≦l≦q₀−1 and for each index iⱼ such that 1≦i₁,i₂, . . . , i_{2q}≦N,

$$\begin{matrix}{I}_{1}^{1} = 1 + {\sum }_{j = 1}^{1} {N}^{1 - j} ( {i}_{j + 2 q - 1} - 1 ) + {\sum }_{j = 1}^{q - 1} {N}^{q - 1} ( {i}_{j} - 1 ) \\ {I}_{2}^{1} = 1 + {\sum }_{j = 1}^{1} {N}^{1 - j} ( {i}_{j + q - 1} - 1 ) + {\sum }_{j = 1}^{q - 1} {N}^{q - 1} ( {i}_{j + q} - 1 )\end{matrix} \vert \qquad ( equation 12 )$$

The multilinearity property of the cumulants and moments makes it possible to express each matrix C_{2q,x}^{l} (q≦1) in a special form.

In fact, under the hypothesis of independence of the sources and noise, the matrix C_{2q,x}^{l} may be expressed, for each 0≦l≦q₀−1, in the following form:

where C_{2q,x}^{l} of the size (N^{q}×N^{q}), C_{2q,z}^{l} of the size (P^{q}×P^{q}) and C_{2q,v}^{l} of the size (N×N) are the 2q order statistical matrices of the vectors x(k), s(k) and v(k), respectively.

δ(.) is the Kronecker symbol and A^{\textcircled{×}q} refers to the matrix A raised to the power of Kronecker where \textcircled{×} represents the Kronecker product operator.

The number l is the same as that defined in equations (11) and (12).

It is important to note that for q≧2, the algebraic structure of the statistical matrix C_{2q,x}^{l} differs from that of the covariance matrix C_{2,x}⁰ used in MUSIC and RapMUSIC algorithms. In addition, the C_{2q,x}^{l} for q=2 is different to the contracted quadricovariance matrix, Qₓ(M), used in the UFO-MUSIC method. In fact if, for q=2 and in the presence of data at real values, the matrix C_{2q,x}^{l} of the size (N²×N²) allows according to equation (13) the following structure:

C_{4,x}⁰=[A(Θ)\textcircled{×}A(Θ)]C_{4,s}⁰[A(Θ)\textcircled{×}A(Θ)]^{T}

the contracted quadricovariance matrix, Qₓ(M), of the size (N×N), allows the following structure:

Qₓ(M)=A(Θ)Qs(B)A(Θ)^{T}

where:

[Qₛ(B)ᵢⱼ=Σ_{r,s=1 . . . p} cum(sᵢ,sⱼ,sₛ,)Bᵣₛ

and:

Bᵣₛ=α(θᵣ)^{T} M(θₛ)

where α(θᵣ) refers to the r-th column vector of the transfer matrix A(Θ) and where the matrix Qₛ(B) has the size (P×P). In this way, the matrix Qₓ(M) has a structure equivalent to that of the covariance matrix C_{2,x}⁰ which gives the authors of UFO-MUSIC the possibility to use it directly in MUSIC and RapMUSIC without needing to modify these two algorithms.

Moreover, it is noted that equation (13) may be re-expressed as follows:

$${C}_{2 q , x}^{l} = {\sum }_{j = 1}^{J} [ \begin{matrix}{A}_{j}^{⊗ q - 1} ⊗ \\ {A}_{j}^{* ⊗ 1}\end{matrix} ] {{C}_{2 q , {3}_{j}}^{1} [ \begin{matrix}{A}_{j}^{⊗ q - 1} ⊗ \\ {A}_{j}^{* ⊗ 1}\end{matrix} ]}^{H} + δ ( q - 1 ) {C}_{2 , y}^{0} \vert , \qquad ( equation 14 )$$

where the matrix C_{2q,sj}^{l}, of the size (Pⱼ^{q}×Pⱼ^{q}), is the 2q order statistical matrix of the vector sⱼ(k).

It should be noted that, in practice, it is not possible to calculate cumulants precisely: they need to be estimated on the basis of the components of the vector x(k).

In fact, firstly, the m (m≦2q) order moments are estimated on the basis of the components of the vectors x(k). The Leonov-Shiryev formula described for real value data in the article by P. McCULLAGH, “Tensor Methods in Statistics”, Chapman and Hall, Monographs on Statistics and Applied Probability, 1987, is then used. This formula establishes the mathematical relationship between the m (m≦2q) order moments and the 2q order cumulants. A similar formula for complex value data and for q belonging to [2,3] is given in the article by L. ALBERA et al., entitled “Blind identification of Overcomplete Mixtures of sources (BIOME)”, Linear Algebra Applications, Special Issue on Linear Algebra in Signal and Image Processing, vol. 391C, pp. 3-30, November 2004.

If the sources of interest and the noise are stationary and ergodic, the m (m≦2q) order moments can be estimated on the basis of the temporal means of the observations, thus making it possible to estimate, via the Leonov-Shiryaev formula, 2q order cumulants.
4. Main Algorithmic Steps in the Method According to an Embodiment of the Invention

As the algorithm “2q-RapMUSIC” (q≧2) of the method according to an embodiment of the invention, using 2q order statistics, should be capable of accounting for potentially (but not completely) consistent, i.e. spatially correlated, signals, it is necessary to make the following hypotheses:
A1) ∀1≦j≦j, Pⱼ<N;
A2) The matrix Aⱼ^{\textcircled{×}q-l}\textcircled{×}Aⱼ^{*\textcircled{×}l} has a full rank Pⱼ^{q};
A3)

$$R ( J , q , 1 ) {=}^{def} {\sum }_{j = 1}^{J} {P}_{j}^{q} < {N}^{q} \vert ;$$

A4) The matrix A(J,q,l)=^{def}[A₁\textcircled{×}^{q-l}\textcircled{×}Aₐ*\textcircled{×}^{l}, . . . , Aⱼ\textcircled{×}^{q-l}\textcircled{×}Aⱼ*\textcircled{×}^{t}]| has the full rank R(J,q,l).

In particular, for P statistically dependent sources, i.e. for which J=1, the hypotheses (A1)−(A2) are therefore reduced to:
A′1) P<N;
A′2) the matrix A(1,q,l)=A^{\textcircled{×}q-l}\textcircled{×}A^{*\textcircled{×}l} has the full rank P^{q}.

Whereas for P statistically independent sources, i.e. for which J=P, the hypotheses (A1)−(A2) are reduced to:
A″1) P<N^{q};
A″2) the matrix A(1,q,f) A^{øq-l}øA^{*øl} has the full rank equal to P;

where ø and A^{øq} represent respectively the operator of the Khatri-Rao product as defined in the article by R. A. HORN and C. R. JOHNSON entitled “Topics in Matrix Analysis, Cambridge University Press, New York, 1999” and A raised to the Khatri-Rao power q.

As such, and as described below, it is possible to define, within the scope of the method according to an embodiment of the invention the 2q order (q≧2) pseudo-spectrum concept, along with the approach referred to by the inventors as “2q-RapMUSIC”.
5. 2q Order (q≧2) Pseudo-Spectrum On the basis of equation (13), it is now possible to calculate the decomposition of the hermitian matrix C_{2q,x}^{l} into eigenvalues:

$${C}_{2 q , x}^{l} = {[ \begin{matrix}{E}_{2 q , x}^{l} & {E}_{2 q , v}^{l}\end{matrix} ] [ \begin{matrix}{L}_{2 q , s}^{l} & 0 \\ 0 & 0\end{matrix} ] [ \begin{matrix}{E}_{2 q , s}^{l} & {E}_{2 q , v}^{l}\end{matrix} ]}^{H} \qquad ( equation 15 )$$

where L_{2q,s}^{l} is the real matrix of the size (R(J,q,l)×R(J,q,l) of the non-null eigenvalues of C_{2q,x}^{l} and E_{2q,s}^{l} the matrix having the size (N^{q}×R(J,q,l)) of the associated orthonormed eigenvectors.

Moreover, E_{2q,v}^{l} is the matrix of the size (N^{q}×(N^{q}−R(J,q,l)) of the orthonormed eigenvectors associated with the null eigenvalues of C_{2q,x}^{l}. As the matrix C_{2q,x}^{l} is hermitian, each column vector of E_{2q,s}^{l} is orthogonal to each column vector of E_{2q,v}^{l}. In addition, vect{A(J,q,l)}=vect{E_{2q,s}^{l}}; consequently, each column vector of A(J,q,l) is orthogonal to each column vector of E_{2q,v}^{l}.

In this way, by referencing as θ_{P}^{j} the vector of the location parameters (position and orientation) of the p-th (1≦p≦P) source, also belonging to the j-th group, and α(θ_{P}^{j})^{\textcircled{×}q-l} \textcircled{×}α(θ_{P}^{j})^{*\textcircled{×}l} the vector located at the “(1−Pⱼ¹)(p−1)/(1−Pⱼ)”-th column of the matrix Aⱼ^{\textcircled{×}q-l}\textcircled{×}Aⱼ^{*\textcircled{×}l}, the vectors α(θ_{P}^{j} )^{\textcircled{×}q-l} \textcircled{×}α(θ_{P}^{j})^{*\textcircled{×}l} (1≦p≦P and 1≦j<J) are all orthogonal to the column vectors of E_{2q,v}^{l}. This result gives us the possibility to construct a cost function wherein the minimization makes it possible to estimate the P vectors θ_{P}^{j}. This cost function, referred to as the 2q order pseudo-spectrum (or pseudo-polyspectrum) is defined by:

J₁(θ)=[α(θ)^{\textcircled{×}q-l}\textcircled{×}α(θ)^{*\textcircled{×}l}]^{H}π_{q,v}^{l}[α(θ)^{\textcircled{×}q-l}\textcircled{×}α(θ)^{*\textcircled{×}l]} (equation 16)

where π_{q,v}^{l}=(E_{2q,v}^{l})^{H}(E_{2q,v}^{l}) is a matrix operator referred to in this case as the 2q order “noise projector”.

It is then preferable to consider the following standardized criterion J₂(θ)=J₁(θ)/∥α(θ)^{\textcircled{×}q-l} \textcircled{×}α(θ)^{*\textcircled{×}l}∥²|0 instead of that defined by equation 16, in order to render the pseudo-polyspectrum constant with respect to θ in the absence of sources.

According to equation (2) and the properties of the Kronecker product, the criterion J₂ may be expressed as follows:

$${J}_{2} ( θ ) = \frac{{Φ}_{q}^{{l}_{11}} {{G}_{q}^{l} ( ρ )}^{H} {\prod }_{q . v}^{l} {G}_{q}^{l} ( ρ ) {Φ}_{q}^{l}}{{Φ}_{q}^{{l}_{11}} {{G}_{q}^{l} ( ρ )}^{H} {G}_{q}^{l} ( ρ ) {Φ}_{q}^{l}} \vert \qquad ( equation 17 )$$

where

$${Φ}_{q}^{l} {=}^{def} {Φ}^{⊗ q - l} ⊗ {Φ}^{* ⊗ l} and {G}_{q}^{l} ( ρ ) {=}^{def} {G ( ρ )}^{⊗ q - l} ⊗ {G ( ρ )}^{* ⊗ l} .$$

Minimizing the criterion J₂ of equation (17) with respect to θ consists of i) minimizing the minimum eigenvalue, referenced λ_{q}^{l} (ρ), of the matrix G_{q}^{l}(ρ)π_{q,v}^{l} G_{q}^{l}(ρ) in the metric G_{q}^{l}(ρ)^{H}G_{q}^{l}(ρ) with respect to ρ such that ρₒₚₜ=min arg{λ_{q}^{l}(ρ)}, and ii) deducing from ρₒₚₜ the minimization solution vector Φ_{q}^{l} of the criterion J₂ with respect to θ and referenced Φ_{q}^{l}(ρₒₚₜ), taking the eigenvector associated with the eigenvalue λ_{q}^{l}(ρₒₚₜ).

In fact, λ_{q}^{l}(ρₒₚₜ) and Φ_{q}^{l}(ρₒₚₜ) verify:

G_{q}^{l}(ρₒₚₜ)^{H}π_{q,v}^{l}G_{q}^{l}(ρₒₚₜ)Φ_{q}^{l}(ρₒₚₜ)=λ_{q}^{l}(ρₒₚₜ)G_{q}^{l}(ρₒₚₜ)^{H}G_{q}^{l}(ρₒₚₜ)Φ_{q}^{l}(ρₒₚₜ) (equation 18)

As a result, the minimization of criterion (17) may partly be obtained by minimizing the following criterion with respect to ρ:

J₃(ρ)=vpm{[G_{q}^{l}(ρ)^{H}G_{q}^{l}(ρ)]⁻¹ G_{q}^{l}(ρ)^{H}π_{q,v}^{l}G_{q}^{l}(ρ)}| (equation 19)

where vpm{B} refers to the minimum specific value of the matrix B. This changes from an optimization in a vectorial subspace of IR⁶, to an optimization in a vectorial subspace of IR³, which offers the advantage of reducing the optimization calculation cost. An additional way to decrease the algorithm calculation cost is to prefer a minimization of the following criterion to that of J₃:

$${J}_{4} ( ρ ) = \frac{det \{ {{G}_{q}^{l} ( ρ )}^{H} {\prod }_{q , v}^{l} {G}_{q}^{l} ( ρ ) \}}{det \{ {{G}_{q}^{l} ( ρ )}^{H} {G}_{q}^{l} ( ρ ) \}} \vert \qquad ( equation 20 )$$

where det{B} refers to the determinant of matrix B.

Thus, it should be noted that, due to these first steps of the method according to an embodiment of the invention, a multi-dimensional search both of the position and orientation was avoided due to the use of criterion J₃ defined by equation (19).

In fact, it is now sufficient to perform an optimization simply with respect to the position vector ρ without worrying about determining the corresponding vector Φ_{q}^{l}, as the latter is then deduced very simply from the optimal position vector determined.

A second improvement then enabled us to reduce the algorithm calculation cost further by replacing the minimization of criterion J₃ by that of criterion J₄. Indeed, it is less expensive to calculate the determinant of a matrix than to decompose same into eigenelements.

We will see in the section below how to extract the orientation vector Φ from the vector Φ_{q}^{l} for all the values of q and l.
6. 2q-RapMUSIC (q≦2) Method

The method referred to as “2q-RapMUSIC” (q≦2) by the inventors will now be described based on i) the factorization of the matrix formulation of the direct problem, ii) the processing of the 2q order (q≦2) virtual network concept via the criterion J₄ defined by equation (20), iii) the use of the extended deflation concept formalized in the 2q order and accounting for the presence of potentially correlated sources.

It is important to note that the deflation principle presented here cannot be interpreted like that of the RapMUSIC method for which the covariance matrix would have been replaced by our 2q order statistical matrix C_{2q,x}^{l}, particularly because the difference in the algebraic structure between both matrices does not allow the use of the orthogonal projector defined in RapMUSIC. In addition, as seen in this section, the definition of the 2q order orthogonal projector is in no way trivial, particularly when the sources of interest are spatially correlated. In addition, the RapMUSIC method works with the signal space and therefore applies its orthogonal projector to the signal space. Our method applies our 2q order projector to the matrix C_{2q,x}^{l} to subsequently calculate the resultant noise space. Our choice to apply the projector before the calculation of the noise space is intended to increase the estimation resolution of the parameters of interest further. Finally, unlike RapMUSIC, we propose here a recursive calculation of the projector, which is less costly in terms of calculation complexity.

The use of higher orders is necessary, as mentioned above, in the processing of under-determined source mixtures or in the presence of Gaussian noise of unknown spatial consistency, or in order to improve the resolution of the estimation of the parameters particularly when the sources are very close.

The need to use a deflation type approach stems from the fact that the noise projector is never perfectly estimated and that the estimation errors necessarily result in determining for the criteria J₁-J₄ not P global minima corresponding to the P sources respectively, but rather P-1 local minima and a global minimum corresponding for example to the source with the highest Signal-To-Noise Ratio (SNR).

Finding the position vector ρ_{ξ(1)} of the source with the highest SNR (signal-to-noise ratio) is easy, and may be performed by determining the minimum of equation (20) on a sufficiently sampled grid of the positions liable to be solutions. Note that ξ(.) is an automorphism of {1,2, . . . ,P}, in other words a permutation of {1,2, . . . ,P}. In fact, the sources can only be located in disorder. However, a glance at equation (1) is sufficient to realize that changing the order in which the components of s(k) and the corresponding columns of A(Θ) are arranged does not change the expression of x(k).

The vector Φ_{q}^{l}(ρ_{ξ(1)}) is obtained similarly to the standardized vector which, multiplied on the left by the matrix G_{q}^{l}(ρ_{ξ(1)}) gives the vector [α(θ_{ξ(1)})^{\textcircled{×}q-l}\textcircled{×}α(θ_{ξ(1)})^{*\textcircled{×}l}.

As described in the previous section, the latter is obtained by taking the eigenvector associated with the minimum eigenvalue of the matrix [G_{q}^{l}(ρ_{ξ(1)})^{H}G_{q}^{l}(ρ_{ξ(1)})]⁻¹ G_{q}^{l}(ρ_{ξ(1)})^{H}π_{q,v}^{l} G_{q}^{l}(ρ_{ξ(1)}).

Then, the vector Φ_{q}^{l} may be extracted from Φ_{q}^{l}(ρ_{ξ(1)}). For this purpose, it is firstly necessary to extract the M=N^{q-2} vectors b_{ξ(1)}^{q,l}(m)(1≦m≦M) of the size (N²×1) (such that Φ_{q}^{l}(ρ_{ξ(1)})=[b_{ξ(1)}^{q,l}(1)^{T}b_{ξ(1)}^{q,l}(2)^{T} . . . b_{ξ(1)}^{q,l}(M)^{T}]^{T}), and then convert them into M matrices B_{ξ(1)}^{q,l}(m) of the size (N×N) (the n-th column of B_{ξ(1)}^{q,l}(m) consists of N consecutive elements of b_{ξ(1)}^{q,l}(m) from the [N(n−1)+1]-nth), and finally jointly diagonalize the set Δ_{ξ(1)}^{q,l} of matrices defined by B_{ξ(1)}^{q,l}(m)B_{ξ(1)}^{q,l}(m)^{H}, B_{ξ(1)}^{q,l}(m)^{T} B_{ξ(1)}^{q,l}(m)*, such that 1≦m≦M, if l=1, by B_{ξ(1)}^{q,l}(m)*, such that 1≦m≦M, if l=1 and otherwise by B_{ξ(1)}^{q,l}(m)^{H}B_{ξ(1)}^{q,l}(m), B_{ξ(1)}^{q,l}(m)*B_{ξ(1)}^{q,l}(m)^{T}, such that 1≦m≦M.

More specifically, the vector Φ_{ξ(1)} is estimated by the common eigenvector for all the matrices of Δ_{ξ(1)}^{q,l}, and associated with the highest eigenvalue, is, within one scale factor, equal to Φ_{ξ(1)}. The term estimation is used as the vector Φ_{ξ(1)} can only be reconstructed within one scale factor, an intrinsic indetermination of the problem. However, note that for the majority of applications, this indetermination does not represent a problem per se. in fact, with respect to our current dipole location problem, the vector Φ_{ξ(1)} is an orientation vector. For this reason, the knowledge of the directing vector, of unit specifications, of Φ_{ξ(1)} is largely sufficient, which is given by the previous estimation.

In terms of algorithms, the joint diagonalization may be performed in our case by means of the JAD (Joint Approximate Diagonalization) method proposed by J.-F. CARDOSO and A. SOLOUMIAC in their article entitled “Jacobi angles for simultaneous diagonalization”, SIAM Journal Matrix Analysis and Applications, vol. 17, no. 1, pp. 161-164, 1996, or the method described by A. YEREDOR in the article entitled “Non-orthogonal joint diagonalization in the least-squares sense with application in blind source separation”, IEEE Transactions On Signal Processing, vol. 50, no. 7, pp. 1545-1553, July 2002.

It is therefore easy to obtain the position vector ρ_{ξ(1)}, and the corresponding orientation vector. It is however more difficult to determine with precision the P−1 local minima of J₄ remaining as the usual minima determination techniques may lack surface minima or not succeed in distinguishing between two minima that are too close together.

The latter problem is resolved by the multi-dimensional parameter identification method according to an embodiment of the invention, by the implementation in any 2q order of the deflation concept, enabling advantageously, moreover, the processing of potentially consistent sources. Note that the term “implementation” particularly refers to a formalization in the 2q order of the deflation concept.

In concrete terms, it consists of withdrawing the contribution of the first source, wherein the location has been estimated, from the observations and running a new global minimum search on the basis of the pseudo-polyspectrum reconstructed using the new observations. This simple and trivial procedure is no less costly as it requires the estimation of P times the statistical matrix of the observations C_{2q,x}^{l}, in an operational context.

A less costly method, in terms of calculation time and resources, consists of withdrawing the contribution of the first estimated source not from the observations but rather from the signal space of the statistical matrix C_{2q,x}^{l}. This solution naturally stems from the approach used in RapMUSIC and requires working with the signal space rather than with the noise space. However, this solution does not allow the possibility of working with the noise space.

Another less costly method, in terms of calculation time and resources, consists of withdrawing the contribution from the first estimated source not from the observations but rather from the statistical matrix C_{2q,x}^{l} itself.

This approach requires the precise study of the algebraic structure of the matrix C^{2q,x}^{l}, so as to cancel the columns of the matrix A^{\textcircled{×}q-l}\textcircled{×}A^{*\textcircled{×}l} involving the vector α(θ_{ξ(1)})^{def}=G(ρ_{μ(1)})φ_{ξ(1)} on the basis of equation (13).

For this purpose, let us consider one of the properties of the Kronecker product: three vectors b,c,d of respective sizes (N_{b}×1), (N_{c}×1), (N_{d}×1), the Kronecker product then verifies b\textcircled{×}c\textcircled{×}d=(I_{N}_{_{b}}\textcircled{×}c\textcircled{×}I_{N}_{_{d}})(b\textcircled{×}d).

In fact, this property makes it possible to demonstrate the following proposal for two matrices ψ_{q,ξ(1)}^{l,m} and Ξ_{q,ξ(1)}^{l,m}, of the size (N^{q}×N^{q}) defined by, for any integer m∈{0,1, . . . , q−1}:

$${Ξ}_{q , ξ ( 1 )}^{l , - 1} = {I}_{{N}^{q}}$$

$${Ψ}_{q , ξ ( 1 )}^{l , m} = \{ \begin{matrix}{Ξ}_{q , ξ ( 1 )}^{l , m - 1} ( {I}_{{N}^{m}} ⊗ a ( {θ}_{ξ ( 1 )} ) ⊗ {I}_{{N}^{q - m - 1}} ) & if m < q - l \\ {Ξ}_{q , ξ ( 1 )}^{l , m - 1} ( {I}_{{N}^{n}} ⊗ {a ( {θ}_{ξ ( 1 )} )}^{*} ⊗ {I}_{{N}^{q - m - 1}} ) & if m \geq q - l\end{matrix} \}$$

$${Ξ}_{q , ξ ( 1 )}^{l , m} = {I}_{{N}^{q}} - {{Ψ}_{q , ξ ( 1 )}^{l , m} [ {( {Ψ}_{q , ξ ( 1 )}^{l , m} )}^{H} {Ψ}_{q , ξ ( q )}^{l , m} ]}^{- 1} {( {Ψ}_{q , ξ ( 1 )}^{l , m} )}^{H}$$

Another procedure, which is also less costly, and different to the approach used in RapMUSIC in the second order, and preferred by the inventors of the present application, firstly consists of withdrawing from C_{2q,x}^{l} the contribution of the first estimated source, and calculating the noise space corresponding to the new resultant statistical matrix. This method enables each deflation to increase the dimension of the noise space and therefore improve the estimation of the parameters of the searched source.

This approach requires the precise study of the algebraic structure of the matrix C_{2q,x}^{l}, particularly via the equation (13). In fact, the equation (13) teaches us that we will be able to withdraw from C_{2q,x}^{l} the contribution of the first estimated source provided that it is possible to cancel the column vectors of the matrix A^{\textcircled{×}q-l}\textcircled{×}A^{*\textcircled{×}l} involving the vector α(θ_{ξ(1)})^{def}=G(ρ_{ξ(1)})φ_{μ(1)}, in other words, the column vectors of A^{\textcircled{×}q-l}\textcircled{×}A^{*\textcircled{×}l} having the form α(θ_{ξ(1)})\textcircled{×}b, b\textcircled{×}α(θ_{ξ(1)}), or b\textcircled{×}α(θ_{ξ(1)})\textcircled{×}c. This is in no way trivial and cannot be deduced from the RapMUSIC deflation procedure.

In this way, it is necessary to construct the matrix Ξ_{q,ξ(1)}^{l,m} of the size (N×N) defined on the basis of the column vector α(θ_{ξ(1)}) as follows:

$${Ξ}_{ξ ( 1 )} = {I}_{N} - \frac{a ( {θ}_{ξ ( 1 )} ) {a ( {θ}_{ξ ( 1 )} )}^{H}}{{a ( {θ}_{ξ ( 1 )} )}^{H} a ( {θ}_{ξ ( 1 )} )} \vert$$

In this case, multiplying the matrix A^{\textcircled{×}q-l}\textcircled{×}A^{*\textcircled{×}l} of the size (N^{q}×P^{q}) on the left by

$${\sum }_{ξ ( 1 )}^{q , l} {=}^{def} {( {Ξ}_{ξ ( 1 )} )}^{⊗ ( q - l )} ⊗ {( {Ξ}_{ξ ( 1 )} )}^{* ⊗ ( q - l )}$$

makes it possible to cancel all the column vectors of A^{\textcircled{×}q-l}\textcircled{×}A^{*\textcircled{×}l} involving α(θ_{ξ(1)}).

The position vector, ρ_{ξ(2)}, of the ξ(2)-th source is then obtained by minimizing criterion J₄ of equation (20) wherein G_{q}^{l}(ρ) is now replaced by

$${Σ}_{ξ ( 1 )}^{q , l} {G}_{q}^{l}$$

(ρ) and where π_{q,v}^{l} will no longer be obtained from the diagonalization of C_{2q,x}^{l}, but rather from the diagonalization of

$${Σ}_{ξ ( 1 )}^{q , l} {{C}_{2 q , x}^{l} ( {Σ}_{ξ ( 1 )}^{q , l} )}^{H} .$$

It is noted that the rank of the matrix

$${Σ}_{ξ ( 1 )}^{q , l} {{C}_{2 q , x}^{l} ( {Σ}_{ξ ( 1 )}^{q , l} )}^{H}$$

is now strictly lower than R(J,q,l). In fact, we decreased the rank of C_{2q,x}^{l} by withdrawing the contribution of the first estimated source from this matrix.

Consequently, the number of specific vectors forming the matrix E_{2q,v}^{l} is increased, which conveys an increase in the size of the noise space, generated by the column vectors of E_{2q,v}^{l}.

Noting that the matrix E_{2q,v}^{l} is directly involved in the calculation of the noise projector π_{q,v}^{l}=(E_{2q,v}^{l})^{H}(E_{2q,v}^{l}), and therefore in the calculation of criterion J₄ of equation (20), we thus offer the possibility to estimate more precisely the parameters of the ξ(2)-th source.

Once the minimization of the new pseudo-polyspectrum is complete, the orientation vector of the ξ(2)-th source, Φ_{ξ(2)}, is extracted according to the procedure detailed above for Φ_{ξ(1)} and the directing vector of the ξ(2)-th source, α(θ_{ξ(2)})^{def}=G(ρ_{ξ(2)})φ_{ξ(2)} can then easily be reconstructed.

The method is then applied by recurrence until the estimation of the P vectors of parameters θ_{P}=[ρ_{P}^{T} Φ_{P}^{%}]^{T} corresponding to the P sources of interest.

It is also specified that the p-th step of the recurrence of the method, or deflation step, according to an embodiment of the invention consists of minimizing the criterion J₄ of equation (20) by replacing G_{q}^{l}(ρ) by

$${Σ}_{ξ ( p )}^{q , l} … {Σ}_{ξ ( 2 )}^{q , l} {Σ}_{ξ ( 1 )}^{q , l} {G}_{q}^{l} ( ρ )$$

and where π_{q,v}^{l} is constructed on the basis of the eigenvectors associated with the null eigenvalues of the matrix

$${Σ}_{ξ ( p )}^{q , l} … {Σ}_{ξ ( 2 )}^{q , l} {Σ}_{ξ ( 1 )}^{q , l} {{C}_{2 q , x}^{l} ( {Σ}_{ξ ( p )}^{q , l} … {Σ}_{ξ ( 2 )}^{q , l} {Σ}_{ξ ( 1 )}^{q , l} )}^{H} .$$

According to another procedure, the matrices

$${\sum }_{ξ ( p )}^{q , l}$$

and Ξ_{ξ(P)} defined by.

$${Σ}_{ξ ( p )}^{q , l} {=}^{def} {( {Ξ}_{ξ ( p )} )}^{⊗ ( q - l )} ⊗ {( {Ξ}_{ξ ( p )} )}^{* ⊗ ( q - l )}$$

and:

Ξ_{ξ(p)}=I_{N}−Ξ_{ξ(p-1}). . . Ξ_{ξ(2)}Ξ_{ξ(1)}α(θ_{ξ,v)})α(θ_{ξ(v)})^{H}(Ξ_{ξ(1)})^{H}(Ξ_{ξ(2)})^{H}. . . (Ξ_{ξ(p-1)})^{H}/∥Ξ_{ξ(p-1)}. . . Ξ_{ξ(2)}Ξ_{ξ(1)}α(Θ_{ξ(p)})∥²

Unlike the deflation operator proposed in equation (13) of the article describing RapMUSIC, the construction of our operator is recursive and thus does not require matrix inversion: this reduces the calculation cost considerably.
7. Identifiability

It is possible to demonstrate that the problem of 2q order processing of P non-Gaussian potentially (but not completely) consistent sources using a network of sensors generating N observations, is, for the arrangement indexed by l, C_{2q,x}^{l}, similar to a second order processing problem of said P sources using a virtual network of N^{q} sensors wherein in general N_{2q}^{l} are different. It is important to note that the number N_{2q}^{l} is dependent implicitly on the algebraic structure of the vector α(θ)=G(ρ)Φ, and more precisely that of the specific gain matrix G(ρ) for each application. For this reason, for fixed q and l values, the number N_{2q}^{l} will change according to the application.

It is deduced from the above results that the maximum number of non-Gaussian and statistically independent, i.e. J=P, sources that can be processed by the “2q-RapMUSIC” algorithm for the indexed arrangement with l is N_{2q}^{l}-d^{q} where d is the size of the vector Φ, in other words, the number of specific quasi-linear parameters for a source.
8. Summary of the Main Successive Steps in the Method According to an Embodiment of the Invention

With reference to FIG. 5, the various steps of the method according to the invention are summarized below, assuming that K temporal samples of surface observations, x(k) (1≦k≦K), are available.

Step 1 (51): Select the suitable statistical order 2q as a function of the number P of sources to be processed. In practice, q is the minimum value ensuring the processing of all the sources potentially present in the multi-dimensional environment studied.

Step 2 (52): Estimate the 2q order cumulants Cᵢ_{₁} _{. . . i}_{_{q}}_{,x}ⁱ^{_{q+1}} ^{. . . i}^{_{2q}} on the basis of the K samples x(k) and select the suitable matrix arrangement for which the estimated 2q order statistical matrix will be referenced Ĉ_{2q,x}^{l}^{ₒₚₜ} it is noted that the size of this statistical matrix is (N^{q}×N^{q})).

Step 3 (53): Calculate the eigenvalues of the hermitian matrix Ĉ_{2q,x}^{l}^{ₒₚₜ} and extract an estimate Ê_{2q,v}^{l}^{ₒₚₜ} opt of the matrix E_{2q,v}^{l} for scenarios where the number of sources and/or their spatial consistency is/are unknown (as such the estimate of P is referenced \hat{P}).

Step 4 (54): Calculate an estimate, \hat{π}_{2q,v}^{l}^{ₒₚₜ}=(Ê_{2q,x}^{l}^{ₒₚₜ})^{H}Ê_{2q,x}^{l}^{ₒₚₜ}, of the 2q order noise projector π_{q,v}^{l}.

Step 5 (55) Calculate an estimate, Ĵ₄, of the criterion J₄ of equation 20 using the matrix π_{2q,v}^{l}^{ₒₚₜ}, for a suitable position grid according to the abovementioned terms, and determine the global minimum thereof, referenced \hat{ρ}_{ξ(P)}.

Step 6 (56): Calculate the vector \hat{φ}_{q}^{l}^{ₒₚₜ}(\hat{ρ}_{ξ(1)}) taking the eigenvector corresponding to the minimum eigenvalue of the matrix ┌G_{q}^{l}^{ₒₚₜ}(\hat{ρ}_{ξ(P)})^{H} G_{q}^{l}^{ₒₚₜ}(\hat{ρ}_{ξ(P)})┐⁻¹ G_{q}^{l}^{ₒₚₜ}(\hat{ρ}_{ξ(P)})^{H} π_{q,v}^{l}^{ₒₚₜ}G_{q}^{l}^{ₒₚₜ}(\hat{ρ}_{ξ(P)}).

Step 7 (57): Extract the vector \hat{φ}_{ξ(1)}, an estimate of the quasi-linear vector Φ_{ξ(1)}, on the basis of the vector \hat{φ}_{q}^{l}^{ₒₚₜ}(\hat{ρ}_{ξ(1)}). For this purpose, first extract the M=Nq−2 vectors \hat{b}_{ξ(1)}^{q,l}^{ₒₚₜ}(m) of the size (N²×1) and convert them into M matrices \hat{B}_{ξ(1)}^{q,l}^{ₒₚₜ}(m) of the size (N×N) and finally calculate the common eigenvector for the M matrices of the set \hat{Δ}_{ξ(1)}^{q,l}^{ₒₚₜ} and associated with the largest eigenvalue. For this purpose, use a joint diagonalization algorithm of matrices such as those mentioned above.

Step 8.1 (581): If the \hat{P} vectors of quasi-linear and non-linear parameters have not yet all been identified, construct the vector

$$a ( {\hat{θ}}_{ξ ( 1 )} ) {=}^{def} G ( {\hat{ρ}}_{ξ ( 1 )} ) {\hat{φ}}_{ξ ( 1 )}$$

and then calculate the matrix \hat{Σ}_{ξ(1)}^{q,l}^{ₒₚₜ} of the size (N^{q}×N^{q}) as described in the previous section by replacing the vector α(θ_{ξ(1)}) by α(\hat{θ}_{ξ(1)}).

Step 8.2 (582): Then reassign the variables as follows:

$$\hat{P} := \hat{P} - 1 ;$$

$${\hat{G}}_{q}^{{l}_{opt}} ( ρ ) := {\hat{Σ}}_{ξ ( 1 )}^{q , {l}_{opt}} {\hat{G}}_{q}^{{l}_{opt}} ( ρ ) ;$$

$${\hat{C}}_{2 q , x}^{{l}_{opt}} := {\hat{Σ}}_{ξ ( 1 )}^{q , {l}_{opt}} {{\hat{C}}_{2 q , x}^{{l}_{opt}} ( {\hat{Σ}}_{ξ ( 1 )}^{q , {l}_{opt}} )}^{H} ;$$

and return to step 3 (53),

Otherwise, go to step 9 (59).

Step 9 (59): If \hat{P}≦N (i.e. if the source mixture is overdetermined), estimate the \hat{P} linear parameters sₚ(k) for each value k by applying to the observation vector x(k) the ASF filter defined by Ŵ=[Ĉ_{2.x}⁰]−1 A(\hat{θ}), where A(\hat{θ}) is the mixture matrix reconstructed on the basis of the estimation \hat{θ} of the quasi-linear and non-linear parameters of the sources using equation (2).

The technical problem that an embodiment of the invention proposes to resolve consists of providing a category of methods, called “2q-RapMUSIC” (q≧2), which is the abbreviation of “2d-D-MUSIC methods”, making it possible simply and effectively to increase the opening and increase the resolution of a network of sensors distributed at specific points of a predetermined multi-dimensional environment, without increasing the number of physical sensors to be used, in order to process and study with greater precision the internal sources of interest in the environment in question and wherein the number is potentially greater than the number of physical sensors used, while remaining insensitive to the presence of a Gaussian noise of unknown spatial consistency.

Such an aim is of particular interest in the biomedical field, and more specifically in that of electrophysiology, with respect to the evaluation of a candidate patient for epilepsy surgery. In fact, the analysis of the data based on the imaging and surface observations (EEG and MEG) does not always make it possible, in view of current techniques, to precisely locate the cerebral zones causing a patient's epileptic attacks, and it is sometimes necessary to study the regions of the brain involved directly using intracerebral electrodes. However, intracerebral electrode implantation is an invasive surgical procedure and therefore very restricting for the patient. In addition, it is a procedure that only a limited number of doctors or neurosurgeons are able to perform, resulting frequently in very long waiting lists for patients.

In this way, an essential aim of an embodiment of the invention, applied to the field of electrophysiology, consists of proposing a novel and inventive technique enabling reliable and precise location without intracerebral electrode implantation, in other words only on the basis of a network of surface sensors, which are fitted in a simple and completely non-invasive manner. Such a technique would also enable pre-surgical examinations by a larger number of doctors and thus make it possible to reduce the waiting lists for patients.

Therefore, another aim of an embodiment of the invention naturally consists of offering such a technique making it possible, solely on the basis of the observations acquired by means of sensors positioned at certain points of the environment in question, to estimate the specific linear, quasi-linear and non-linear parameters for the internal sources of interest in said environment, so as to be able to monitor and be able to understand both normal behaviors thereof (in the biomedical field, studying the cerebral function in a subject's sleep phases, for example) and abnormal behaviors thereof (in seismology, detecting the epicenter of an earthquake or monitoring the propagation thereof, or in the biomedical field, detecting and monitoring an epileptic attack, for example), without impairing the quality of the results obtained compared with standard methods, for example those using intracerebral electrodes in electrophysiology.

A further aim of an embodiment of the invention is to provide such a technique which makes it possible to use the 2q (q≧2) order virtual network theory, and/or, unlike the UFO-MUSIC method, the algebraic structure of the 2q (q≧2) order cumulant tensor overall and not that of matrix sections of said tensor, in order to be able to simultaneously i) resolve a poorly posed problem with respect to the identification of quasi-linear and non-linear parameters, ii) handle a Gaussian type noise of unknown spatial consistency, and iii) increase the resolution of the estimation of linear, quasi-linear and non-linear parameters in an overdetermined context.

A further aim of an embodiment of the invention is to offer such a technique capable of using a possible factorization of the matrix formulation of the direct problem so as to dissociate the estimation of the non-linear parameters of the sources from the estimation of the quasi-linear parameters, and thus reduce the calculation costs substantially when the quasi-linear parameters are unknown. Therefore, this aim is intended to guarantee the feasibility of the method in an operational context when the quasi-linear parameters of the sources are unknown. A further aim is to provide such a technique making it possible to use the extended deflation concept formalized in the 2q (q≧2) order by the authors, in order to increase the resolution of the estimation of the parameters.

Another aim is to provide such an invention making it possible to handle sources with potentially (but not completely) correlated linear (time course) parameters, particularly in space.

A further aim of an embodiment of the invention is to provide such a technique not using any other prospective information on the linear parameters of the sources.

A final aim of an embodiment of the invention consists of providing such a technique which is, on one hand, inexpensive and, on the other, relatively simple to implement, including in devices, for example of the electroencephalography, magnetoencephalography, seismic study or exploration type, or any type of device dedicated to the study of the activity of a given multi-dimensional environment, on the basis of observation data acquired at certain points thereof.

Although the present disclosure has been described with reference to one or more examples, workers skilled in the art will recognize that changes may be made in form and detail without departing from the scope of the disclosure and/or the appended claims.

1. Identification method of multi-dimensional, linear, quasi-linear and non-linear type parameters, associated with a plurality of P≧1 sources of interest present in a predetermined multi-dimensional environment, by a plurality of observations in a finite number N≧1, obtained using physical sensors organized in the form of a network of sensors distributed at pre-defined points of said environment, wherein said identification method comprises at least the following steps:
recording of physical measurements making it possible to produce at least one vector of N observations generated by a mixture of linear parameters, representative of P sources of interest, and an additional noise;
construction, on the basis of said at least one observation vector, of a 2q (q≧2) order statistical matrix of the size (N^{q}×N^{q}); and
estimation of at least one first multi-dimensional parameter of said P sources of interest by the estimate of at least one second multi-dimensional parameter.

2. Identification method of multi-dimensional parameters of sources of interest according to claim 1, wherein the method implements a location and reconstruction step of the electrical activity generated by the plurality of P≧1 sources of interest, representative of electric current sources, modeled in the form of electric current dipoles, referred to as dipolar sources, when said predetermined multi-dimensional environment is a conducting volume, and wherein the location and reconstruction steps account for the plurality of the finite number of said N observations.

3. Identification method of multi-dimensional parameters of sources of interest according to claim 2, wherein linear, quasi-linear and non-linear parameters are respectively representative of the time courses or dipolar moments, the orientation and position parameters of each of the electric current sources.

4. Identification method of multi-dimensional parameters of sources of interest according to claim 1, wherein, for any time k, the observation vector of the length N is expressed in the following form: x(k)=A(Θ)s(k)+v(k) where:
s(k) is a vector, of the size (P×1), representative of the linear parameters corresponding to the time courses of said P sources of interest, which are non-Gaussian and potentially (but not completely) correlated according to said at least one first multi-dimensional parameter;
A(Θ) is an instantaneous mixture matrix, of the size (N×P), where Θ={Θ_{1 . . . ,} Θ_{P}} is the set of the P vectors of quasi-linear and non-linear parameters of the sources of interest and where each of the P column vectors of A(Θ) is broken down in the form: α(Θ_{P})=G(ρ_{P})Φ_{P} where ρ_{P} and Φ_{P} represent respectively the non-linear parameters, on one hand, and quasi-linear parameters, on the other, associated with the p-th source of interest, the mixture matrix defining a transfer function between the P sources of interest and the N observations, and;
v(k) is the vector, of the size (N×1), of the additional noise, independent from said sources of interest.

5. Identification method of multi-dimensional parameters of sources of interest according to claim 1, wherein the method comprises at least one step i) consisting of estimating the 2q order cumulants Cᵢ_{ᵢ} _{. . . i}_{_{q}}_{,x}ⁱ^{_{q+1}} ^{. . . i}^{_{2q}} on the basis of the K samples x(k), ii) consisting of selecting the suitable matrix arrangement for which the estimated 2q order statistical matrix, of the size (N^{q}×N^{q}), will be referenced Ĉ_{2q,x}^{l}^{ₒₚₜ}.

6. Identification method of multi-dimensional parameters of sources of interest according to claim 1, wherein the method comprises at least one estimation step of the rank of said estimated matrix Ĉ_{2q,x}^{l}^{ₒₚₜ}, and the number P of sources involved.

7. Identification method of multi-dimensional parameters of sources of interest according to claim 6, wherein the method comprises at least one eigenvalues decomposition step of the matrix Ĉ_{2q,x}^{l}^{ₒₚₜ} and a construction step of a cost function, referred to as a 2q order pseudo-spectrum or pseudo-polyspectrum, along with a minimization step of said cost function to estimate each of said \hat{P} vectors of quasi-linear and non-linear parameters associated with each of said \hat{P} sources of interest, where, \hat{P} is the estimate of P.

8. Identification method of multi-dimensional parameters of sources of interest according to claim 7, wherein said cost function is expressed in the form
$${\hat{J}}_{4} ( ρ ) = \frac{det \{ {{G}_{q}^{l} ( ρ )}^{H} {\hat{Π}}_{q , v}^{l} {G}_{q}^{l} ( ρ ) \}}{det \{ {{G}_{q}^{l} ( ρ )}^{H} {G}_{q}^{l} ( ρ ) \}} ,$$
where:
$${\hat{Π}}_{q , v}^{{l}_{opt}} = {( {\hat{E}}_{2 q , v}^{{l}_{opt}} )}^{H} {\hat{E}}_{2 q , v}^{{l}_{opt}}$$
is a matrix operator, referred to as the 2q order noise projectors, where Ê_{2q,v}^{l}^{ₒₚₜ} is the matrix of the orthonormed eigenvectors associated with the null eigenvalues of said matrix Ĉ_{2q,x}^{l}^{ₒₚₜ};
$${G}_{q}^{l} ( ρ ) {=}^{def} {G ( ρ )}^{⊗ q - l} ⊗ {G ( ρ )}^{* ⊗ l}$$
where G(ρ) is the function gain matrix of the non-linear parameter vector ρ of the size (N×L), where L is the length of the quasi-linear parameter vector Φ.

9. Identification method of multi-dimensional parameters of sources of interest according to claim 7, wherein the method implements a cost function minimization step, performed on the basis of a 2q (q>1) order deflation method representative of recursive estimation of each of said \hat{P} vectors of quasi-linear and non-linear parameters associated with each of the \hat{P} sources of interest.

10. Identification method of multi-dimensional parameters of sources of interest according to claim 9, wherein the p-th step (1≦p≦P) of the recursive procedure comprises at least one of the following steps:
determination of the global minimum cost function, where the estimate is referenced \hat{ρ}_{ξ(P)};
calculation of a vector \hat{φ}_{q}^{l}^{ₒₚₜ}(\hat{ρ}_{ξ(P)}) taking the eigenvector corresponding to the minimum eigenvalue of the matrix ┌G_{q}^{l}^{ₒₚₜ}(\hat{ρ}_{ξ(P)})^{H}G_{q}^{l}^{ₒₚₜ}(\hat{ρ}_{ξ(P)})┐⁻¹ g_{q}^{l}^{ₒₚₜ}(\hat{ρ}_{ξ(P)})^{H}π_{q,v}^{l}^{ₒₚₜ}G_{q}^{l}^{ₒₚₜ}(\hat{ρ}_{ξ(P)});
extraction of a vector \hat{φ}_{ξ(P)} representing an estimate of the nuisance parameter vector Φ_{ξ(P)}, on the basis of the vector \hat{φ}_{q}^{l}^{ₒₚₜ}(\hat{ρ}_{ξ(P)});
in the event at least one source remaining wherein the quasi-linear and non-linear parameters have not yet been identified, i) construction of a vector α(\hat{θ}_{ξ(P)})^{def}=G(\hat{ρ}_{ξ(p)})\hat{φ}_{ξ(p)}, and ii) calculation of a matrix
$${\hat{Σ}}_{ξ ( p )}^{q , {l}_{opt}}$$
of the size (N^{q}×N^{q}) accounting for a replacement of said vector α(θ_{ξ(P)}) by said vector αa(\hat{θ}_{ξ(P)}) and iii) reallocation of the variables according to the following functions:
$$\begin{matrix}\hat{ P} := \hat{P} - 1 ; \\ {\hat{G}}_{q}^{{l}_{opt}} ( ρ ) := {\hat{Σ}}_{ξ ( 1 )}^{q , {l}_{opt}} {\hat{G}}_{q}^{{l}_{opt}} ( ρ ) ; \\ {\hat{C}}_{2 q , x}^{{l}_{opt}} := {\hat{Σ}}_{ξ ( 1 )}^{q , {l}_{opt}} {{\hat{C}}_{2 q , x}^{{l}_{opt}} ( {\hat{Σ}}_{ξ ( 1 )}^{q , {l}_{opt}} )}^{H} ;\end{matrix}$$

11. Identification method of multi-dimensional parameters of sources of interest according to claim 10, wherein said extraction step of a vector \hat{φ}_{ξ(P)} comprises the following sub-steps:
extraction of M=N^{q-2} vectors \hat{b}_{ξ(P)}^{q,l}^{ₒₚₜ}(m) of the size (N²−1);
conversion of the vectors into M matrices \hat{B}_{ξ(P)}^{q,l}^{ₒₚₜ}(m) of the size (N×N);
calculation of a common eigenvector for the M matrices of the set \hat{Δ}_{ξ(P)}^{q,l}^{ₒₚₜ} associated with the largest eigenvalue.

12. Identification method of multi-dimensional parameters of sources of interest according to claim 1, wherein said P sources of interest are less than the number in the observations, and the method implements an ASF (“Alternating Sequential Filtering”) filter construction step defined by the formula Ŵ=[Ĉ_{2,x}⁰]⁻¹ A(\hat{Θ}), where A(\hat{Θ}) is the mixture matrix reconstructed from the estimation \hat{Θ} of said quasi-linear and non-linear parameters of the sources of interest, in order to estimate the linear parameters thereof.

13. Identification device of multi-dimensional parameters of sources of interest, wherein the device comprises:
a processor suitable for implementing steps of an identification method of multi-dimensional, linear, quasi-linear and non-linear type parameters, associated with a plurality of P≧1 sources of interest present in a predetermined multi-dimensional environment, by a plurality of observations in a finite number N≧1, obtained using physical sensors organized in the form of a network of sensors distributed at pre-defined points of said environment, wherein said identification method comprises at least the following steps:
recording of physical measurements making it possible to produce at least one vector of N observations generated by a mixture of linear parameters, representative of P sources of interest, and an additional noise;
construction, on the basis of said at least one observation vector, of a 2q (q≧2) order statistical matrix of the size (N^{q}×N^{q}); and
estimation of at least one first multi-dimensional parameter of said P sources of interest by the estimate of at least one second multi-dimensional parameter.

14. Electroencephalography and/or magnetoencephalography type device, wherein the device comprises an identification device of multi-dimensional parameters of sources of interest according to claim 13.

15. Computer program product stored on a computer-readable medium, such program comprising program code instructions for the implementation of steps of an identification method of multi-dimensional parameters, of the linear, quasi-linear and non-linear type, associated with a plurality of P≧1 sources of interest present in a predetermined multi-dimensional environment, by a plurality of observations in a finite number N≧1, obtained using physical sensors organized in the form of a network of sensors distributed at pre-defined points of said environment, wherein said identification method comprises at least the following steps:
recording of physical measurements making it possible to produce at least one vector of N observations generated by a mixture of linear parameters, representative of P sources of interest, and an additional noise;
construction, on the basis of said at least one observation vector, of a 2q (q≧2) order statistical matrix of the size (N^{q}×N^{q}); and
estimation of at least one first multi-dimensional parameter of said P sources of interest by the estimate of at least one second multi-dimensional parameter.

16. Application of the identification method of multi-dimensional parameters, of the linear, quasi-linear and non-linear type, associated with a plurality of P≧1 sources of interest present in a predetermined multi-dimensional environment, by a plurality of observations in a finite number N≧1, according claim 1, in a field belonging the group comprising:
electroencephalography;
magnetoencephalography;
geophysics;
seismology.
